(19) United States
(12) Patent Application Publication
(10) Pub. No.: US 2023/0077983 A1
(43) Pub. Date: Mar. 16, 2023

(54) METHODS OF TREATING AND PREVENTING ENGRAFTMENT ARRHYTHMIAS

(71) Applicants: UNIVERSITY OF WASHINGTON, Seattle, WA (US); VANDERBILT UNIVERSITY, Nashvillel, TN (US)

(72) Inventors: Robert Scott THIES, Seattle, WA (US); William Robb MACLELLAN, Seattle, WA (US); Kenta NAKAMURA, Seattle, WA (US); Charles E. MURRY, Seattle, WA (US); Bjorn KNOLLMANN, Nashville, TN (US)

(73) Assignees: UNIVERSITY OF WASHINGTON, Seattle, WA (US); VANDERBILT UNIVERSITY, Nashville, TN (US)

(21) Appl. No.: 17/798,127

(22) PCT Filed: Feb. 9, 2021
(86) PCT No.: PCT/US2021/017220

§ 371 Date: Aug. 8, 2022

Related U.S. Application Data
(60) Provisional application No. 62/972,330, filed on Feb. 10, 2020.

(51) Int. Cl. A61K 31/343; A61K 31/55; C12N 5/077; A61K 35/34; A61K 9/00; A61P 9/00

(52) U.S. Cl. CPC A61K 31/343; A61K 31/55; C12N 5/0657; A61K 35/34; A61K 9/0053; A61K 9/0019; A61P 9/00

(57) ABSTRACT

Described herein are methods and compositions related to treating and preventing an engraftment arrhythmia with an effective amount of amiodarone and ivabradine. Also described herein is a method of cardiomyocyte transplant, the method comprises: dministering in vitro-differentiated cardiomyocytes to cardiac tissue of a subject in need thereof; and administering to the subject an amount of amiodarone and an amount of ivabradine effective to reduce engraftment arrhythmia in the subject.

### CROSS-REFERENCE TO RELATED APPLICATIONS

This application claims benefit under 35 U.S.C. § 119(e) of U.S. Provisional Application No. 62/972,330 filed Feb. 10, 2020, the contents of which are incorporated herein by reference in their entirety.

### TECHNICAL FIELD

The technology described herein relates to methods of treating cardiovascular disease.

### BACKGROUND

Cardiovascular disease remains the leading cause of death for both men and women worldwide, with a rapidly growing impact on developing nations. Cardiomyocyte replacement therapy is an area of active investigation for the treatment of cardiovascular disease, and can restore heart function after myocardial infarction. Human stem cells cultured in vitro can serve as a starting material for producing human cardiomyocytes for engraftment into an injured heart. However, there are complications involved with cardiac engraftment, one of which is the lack of maturity of the in vitro-differentiated cardiomyocytes which can lead to the development of transient cardiac arrhythmias. A method of preventing and treating arrhythmias caused by the cardiac graft is needed to improve patient outcomes following cardiomyocyte replacement therapy.

### SUMMARY

The methods described herein are related, in part, to the discovery that the combination of amiodarone and ivabradine treatment improves survival and reduces arrhythmia burden in subjects receiving cardiac grafts. The methods described herein reduce the transient graft-associated arrhythmias and significantly improve the safety and survival of subjects treated with both anti-arrhythmic agents.

In one aspect, described herein is a method of treating or ameliorating an engraftment arrhythmia in a subject recipient of a cardiac graft of cardiomyocytes, the method comprising administering to the subject an effective amount of amiodarone and an effective amount of ivabradine.

In one embodiment of this or any other aspect, the cardiac graft of cardiomyocytes comprises in vitro-differentiated cardiomyocytes. In another embodiment, the in vitro-differentiated cardiomyocytes are differentiated from induced pluripotent stem (iPS) cells or from embryonic stem (ES) cells.

In another embodiment of this or any other aspect, the cardiac graft of cardiomyocytes is derived from stem cells autologous to the subject.

In another embodiment of this or any other aspect, the cardiac graft of cardiomyocytes is derived from stem cells allogeneic to the subject.

In another embodiment of this or any other aspect, the amiodarone and ivabradine are administered concurrently with the cardiac graft of cardiomyocytes.

In another embodiment of this or any other aspect, administration of amiodarone is initiated prior to administration of the graft of cardiomyocytes. In one embodiment, administration can be, for example 1 day before, 2 days before, 3 days before, 4 days before, 5 days before, 6 days before, or 7 days or more prior to the graft.

In another embodiment of this or any other aspect, administration of ivabradine is initiated prior to administration of the graft of cardiomyocytes. In one embodiment, administration can be, for example 1 day before, 2 days before, 3 days before, 4 days before, 5 days before, 6 days before, or 7 days or more prior to the graft.

In another embodiment of this or any other aspect, administration of both amiodarone and ivabradine is initiated prior to administration of the cardiac graft of cardiomyocytes. In one embodiment, administration can be, for example 1 day before, 2 days before, 3 days before, 4 days before, 5 days before, 6 days before, or 7 days or more prior to the graft. In another embodiment, amiodarone can be administered sooner than ivabradine, e.g., 1 day, 2 days, 3 days, 4 days, 5 days, or 6 days sooner.

In another embodiment of this or any other aspect, the administration of ivabradine is initiated concurrently with or after administration of the cardiac graft of cardiomyocytes.

In another embodiment of this or any other aspect, the administration of amiodarone is initiated concurrently with or after administration of the cardiac graft of cardiomyocytes.

In another embodiment of this or any other aspect, the administration of amiodarone is a single bolus administration.

In another embodiment of this or any other aspect, the administration is continuous or repeated administration.

In another embodiment of this or any other aspect, the administration is oral administration and/or intravenous injection.

In another embodiment of this or any other aspect, the amiodarone is administered orally at a dose of 100-800 mg, three times per day.

In another embodiment of this or any other aspect, the amiodarone is administered by IV bolus at a dose of 100-300 mg.

In another embodiment of this or any other aspect, the amiodarone is administered to a serum concentration of 1.5 to 2.5 μg/ml.

In another embodiment of this or any other aspect, the ivabradine is orally administered at a dose of 5 to 15 mg, twice per day.

In another embodiment of this or any other aspect, the ivabradine is administered when there is tachycardia.

In another embodiment of this or any other aspect, ivabradine is administered to maintain a resting heart rate of less than or equal to 150 beats per minute (bpm). In another embodiment, ivabradine is administered to maintain a resting heart rate of 60 to 150 bpm, e.g., 60-140 bpm, 60-130 bpm, 60-120 bpm, 60-110 bpm, 60-100 bpm, 60-90 bpm or 60-80 bpm.

In another embodiment of this or any other aspect, the administration of amiodarone and ivabradine reduces post-graft accelerated heart rate experienced by the graft recipient by at least 10% relative to a subject receiving a graft of the same type of cells in the absence of amiodarone and ivabradine administration.

In another embodiment of this or any other aspect, the administration of amiodarone and ivabradine reduces the proportion of time in which the subject experiences engraftment arrhythmia by at least 10% relative to a subject receiving a graft of the same type of cardiomyocytes in the absence of amiodarone and ivabradine administration. In another embodiment, the proportion of time in which the subject experiences engraftment arrhythmia is reduced by at least 20%, at least 30%, at least 40%, at least 50%, at least 60%, at least 70%, at least 80%, at least 90% or more.

In another aspect, described herein is a method of cardiomyocyte transplant, the method comprising: a) administering in vitro-differentiated cardiomyocytes to cardiac tissue of a subject in need thereof; and b) administering to the subject an amount of amiodarone and an amount of ivabradine effective to reduce engraftment arrhythmia in the subject.

In one embodiment of this or any other aspect, engraftment arrhythmia is reduced in the subject relative to a subject receiving in vitro-differentiated cardiomyocytes without receiving amiodarone and ivabradine.

In another embodiment of this or any other aspect, the in vitro-differentiated cardiomyocytes are differentiated in vitro from embryonic stem cells or from iPS cells.

In another embodiment of this or any other aspect, the iPS cells are autologous to the subject.

In another embodiment of this or any other aspect, the iPS cells are allogeneic to the subject.

In another embodiment of this or any other aspect, the amiodarone and ivabradine are administered concurrently with the in vitro-differentiated cardiomyocytes.

In another embodiment of this or any other aspect, administration of amiodarone is initiated prior to administration of the in vitro-differentiated cardiomyocytes.

In another embodiment of this or any other aspect, administration of ivabradine is initiated prior to administration of the in vitro-differentiated cardiomyocytes.

In another embodiment of this or any other aspect, administration of both amiodarone and ivabradine is initiated prior to administration of the in vitro-differentiated cardiomyocytes.

In another embodiment of this or any other aspect, the administration of ivabradine is initiated concurrently with or after administration of the in vitro-differentiated cardiomyocytes.

In another embodiment of this or any other aspect, the administration of amiodarone is initiated concurrently with or after administration of the in vitro-differentiated cardiomyocytes.

In another embodiment of this or any other aspect, the administration of ivabradine is a single bolus administration.

In another embodiment of this or any other aspect, the administration is continuous or repeated administration.

In another embodiment of this or any other aspect, the administration of amiodarone and ivabradine is short-term.

In another embodiment of this or any other aspect, the administration of amiodarone and ivabradine is terminated after engraftment arrhythmia burden reaches zero, without recrudescence of the arrhythmia.

In another embodiment of this or any other aspect, the administration is oral administration and/or intravenous (IV) injection.

In another embodiment of this or any other aspect, the amiodarone is administered orally at a dose of 100-800 mg, three times per day.

In another embodiment of this or any other aspect, the amiodarone is administered by IV bolus at a dose of 100-300 mg.

In another embodiment of this or any other aspect, the amiodarone is administered to a serum concentration of 1.5 to 2.5 μg/ml.

In another embodiment of this or any other aspect, the ivabradine is orally administered at a dose of 5 to 15 mg, twice per day.

In another embodiment of this or any other aspect, the ivabradine is administered when there is tachycardia.

In another embodiment of this or any other aspect, ivabradine is administered to maintain a resting heart rate of less than or equal to 150 beats per minute (bpm).

In another embodiment of this or any other aspect, about 10 million cardiomyocytes to about 10 billion cardiomyocytes are administered to the subject.

In another embodiment of this or any other aspect, the subject is a human.

In another embodiment of this or any other aspect, the subject has or is at risk for having a cardiovascular disease or a cardiac event. In another embodiment, the cardiovascular disease or the cardiac event is selected from the group consisting of: atherosclerotic heart disease, myocardial infarction, cardiomyopathy, cardiac arrhythmia, valvular stenosis, congenital heart disease, chronic heart failure, regurgitation, ischemia, fibrillation, and polymorphic ventricular tachycardia.

In another aspect, described herein is a composition comprising in vitro-differentiated cardiomyocytes, amiodarone and ivabradine.

### BRIEF DESCRIPTION OF THE DRAWINGS

FIGS. 1A-1B demonstrate that amiodarone and ivabradine therapy reduces heart rate and arrhythmia burden in pigs that received cardiac grafts compared with untreated pigs that received cardiac grafts. FIG. 1A shows the average heart rate for pigs that received amiodarone/ivabradine treatment (black squares, amiodarone ±ivabradine, n=6) compared with untreated pigs (white circles, No anti-arrhythmic, n=6) over the course of engraftment therapy (days). FIG. 1B shows the average percentage time in arrhythmia observed for pigs that received amiodarone/ivabradine treatment (black squares, amiodarone ±ivabradine, n=6) compared with untreated pigs (white circles, No anti-arrhythmic, n=6) over the course of engraftment therapy (days).

FIG. 2 shows the percentage of cardiac survival in treated (solid black line, amiodarone ivabradine, n=6) and untreated pigs (dashed lines, No anti-arrhythmic, n=6) with cardiac grafts.

FIGS. 3-14 show the heart rate and % arrhythmia for each individual pig that received cardiac graft in the presence and absence of amiodarone/ivabradine treatment. Heart rate, reported as beats per minute, is indicated in grey on the left vertical axis in the figures. Arrhythmia burden, reported as percent time in arrhythmia compared to normal sinus rhythm, is indicated by the black circles on the right vertical axis.

The pigs treated with anti-arrhythmic agents are shown in FIGS. 3-8. Amiodarone treatment is indicated by the black bar at the top of each graph, while ivabradine treatment is indicated by the checked bar.

Pigs that received a cardiac graft but did not receive anti-arrhythmic treatment are shown in FIGS. 9-14.

FIG. 15 shows an embodiment of anti-arrhythmic regimen doses for pigs (swine) and humans. BID: Twice per day; PO: Oral Administration.

FIG. 16 shows a flowchart of the study design. Phase 1 consisted of nine total subjects, four used to study the natural history of engraftment arrythmia (EA) and five used to screen seven candidate antiarrhythmic agents. Amiodarone and ivabradine were found to have promising signs of effect and advanced for further study. Phase 2 consisted of 19 total subjects: nine assigned to treatment with amiodarone and ivabradine, eight to no treatment and two to infarct with sham transplant and no anti-arrhythmic drug treatment.

FIG. 17 shows a study timeline for Phase 2 drug trial of chronic amiodarone and adjunctive ivabradine therapy. Myocardial infarction (MI) was induced by 90-minute balloon occlusion of the mid-left anterior descending artery two weeks prior to human embryonic stem cell-derived cardiomyocyte transplantation (day 0). All subjects received multi-drug immunosuppression. Treatment cohort received rate and rhythm control with combined oral amiodarone and adjunctive oral ivabradine.

FIG. 18 shows plasma amiodarone levels in pigs. Amiodarone levels were measured in plasma by a liquid chromatography-mass spectrometry assay. Chronic oral amiodarone in six pigs was discontinued after achieving electrical maturation and stabilization of engraftment arrythmia. Serum concentrations of amiodarone were assayed weekly, including 3-4 weeks after discontinuation.

FIG. 19 shows variable morphologies of engraftment arrhythmia (EA) in a single pig.

Examples of normal sinus rhythm (NSR) and three morphologies of EA resembling accelerated junctional rhythm (AJR), ventricular tachycardia (VT) and accelerated idioventricular rhythm (AIVR) are observed in this single pig (subject 12). Note the variation in rate, electrical axis, and QRS duration. A continuous rhythm recording exhibits multiple foci of impulse generation from hESC-CM grafts interacting at various levels of the host conduction system to induce EA. No sustained arrythmias were noted in surgical sham controls.

FIG. 20 shows hESC-CM graft histology and location. Left panel: Histological sections stained with picrosirius red to identify collagen (infarct) and fast green to identify viable myocardium. Adjacent sections labeled with human cTnT identify transplanted hESC-CM graft within unstained porcine myocardium and scar tissue. Both treatment and no treatment sections were obtained on post-transplantation day 42. Right panel: Transplanted hESC-CM grafts were located similarly between treatment (closed square) and no treatment (open circle) and successfully targeted the infarct and pen-infarct regions of the anterior wall.

FIG. 21A-21B demonstrates the acute effects of amiodarone and ivabradine on engraftment arrhythmia. Amiodarone was effective as an intravenous bolus to cardiovert engraftment arrythmia to normal sinus or a lower heart rate (FIG. 21A). Ivabradine administered orally significantly slowed EA but did not cardiovert (FIG. 21B). These data support a combined amiodarone and ivabradine antiarrhythmic strategy for rhythm and rate control of EA.

FIG. 22A-22B shows antiarrhythmic treatment with amiodarone and ivabradine for engraftment arrhythmia in pig. (FIG. 22A) Kaplan-Meier curve for freedom from primary outcome of cardiac death, unstable EA or heart failure was significantly improved with treatment compared to no treatment (p=0.002). Tic marks on treatment line indicate non-cardiac death due to opportunistic infection (days 19 and 26) or a planned euthanasia (day 30). (FIG. 22B) Kaplan-Meier curve for overall survival shows statistically borderline improvement with treatment compared to no treatment (p=0.051). *Death due to Pneumocystis pneumonia. **Death due to porcine cytomegalovirus. Abbreviations: CI, 95% confidence interval.

FIG. 23A-23F demonstrates the effect of antiarrhythmic treatment on heart rate and arrhythmia burden. Pooled daily average heart rate (FIG. 23A) and pooled daily average arrhythmia burden (FIG. 23B) with treatment (black) compared to no treatment (gray). No significant difference in heart rate or arrhythmia burden between treatment and no treatment was observed on day 30 post-transplantation. Sham transplant (light grey) did not induce tachycardia or arrythmia. Subject-level averaged daily heart rate (FIG. 23C) and arrhythmia burden (FIG. 23D) for antiarrhythmic treatment (black), no treatment (light grey) and sham transplant (grey). Unexpected death or euthanasia denoted by black symbol. Peak heart rate (FIG. 23E) and peak arrythmia burden (FIG. 23F) were significantly reduced with treatment (black) compared to no treatment (light gray). * p<0.05, ** p<0.005.

FIG. 24A-24B shows that transplanted hESC-CM graft interacts with a diffuse Purkinje conduction system in the porcine myocardium. Purkinje fibers are distributed in a mesh-like network throughout the native porcine myocardium (FIG. 24A). Subendocardial and intramyocardial connexin 40 (Cx40)+Purkinje fibers (PFs, white) in transverse section of left ventricular free wall, scale bars 2 mm. Intramyocardial PFs are shown with higher magnification insets. Further magnified view of white boxed regions show Cx40 localizes to gap junctions of Purkinje cells that display lower sarcomere content (F-Actin) (i.) and lack T-Tubules (WGA) (ii.) in contrast to surrounding cardiomyocytes, scale bar 20 μm. hPSC-cardiomyocyte graft marked by human-specific slow skeletal cardiac troponin I (ssTnI) interact with Cx40+(white) PFs (FIG. 24B). High magnification of boxed regions show example of Purkinje-transitional cell-graft (i., white arrowhead) and direct Purkinje-graft (ii., white arrow) interactions, scale bar 500 μm (top) or 50 μm (bottom).

FIG. 25 demonstrates that connexin 40 specifically stains Purkinje fibers. Connexin 40 (Cx40) marks Purkinje fibers (PFs) and localizes to gap junctions of PFs that display lower sarcomere content (F-Actin) and lack T-Tubules (WGA) in contrast to surrounding cardiomyocytes, scale bars 20 μm.

### DETAILED DESCRIPTION

One of the major challenges in cardiac cell replacement therapy for the treatment of cardiovascular diseases, is that human stem cell-derived cardiomyocytes (ES and iPS cardiomyocytes) lack the functional maturity to fully integrate with the native heart tissue. As a result, graft-induced arrhythmias appear shortly after the in vitro-differentiated cardiomyocytes are transplanted, during which time the recipient is at risk for sudden cardiac death and heart failure. After a few weeks, subjects that do survive the engraftment procedure have an observed return to normal sinus rhythm that reflects the in vivo maturation of the transplanted cardiomyocytes. In order for this to occur, sufficient electrical integration with the host myocardium is necessary to avoid arrhythmogenicity and improve survival following cardiac engraftment.

The methods described herein are related, in part, to the discovery that the combination of amiodarone and ivabradine treatment improves survival, prevents tachycardia and reduces arrhythmia burden in subjects that receive cardiac grafts. The methods described herein reduce the transient graft-associated arrhythmias and significantly improve the safety and survival of subjects treated with both anti-arrhythmic agents.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in cellular and molecular biology, and biochemistry can be found in The Merck Manual of Diagnosis and Therapy, 19th Edition, published by Merck Sharp & Dohme Corp., 2011 (ISBN 978-O-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

“Treatment” of a cardiac disorder, a cardiac disease, event, or a cardiac injury (e.g., myocardial infarction) as referred to herein refers to therapeutic intervention that enhances cardiac function, reduces engraftment arrhythmia, and/or enhances cardiomyocyte engraftment and/or enhances cardiomyocyte transplant or graft vascularization in a treated area, thus improving the function of e.g., the heart. That is, cardiac “treatment” is oriented to the function of the heart (e.g., enhanced function within an infarcted area), and/or other site treated with the compositions described herein. A therapeutic approach that improves the function of the heart, for example as assessed by measuring heart rate and/or the frequency of arrhythmias reduced by at least 10%, and preferably by at least 20%, 30%, 40%, 50%, 75%, 90%, 100% or more, e.g., 2-fold, 5-fold, 10-fold or more, up to and including full function, relative to an appropriate control is considered effective treatment. Effective treatment need not cure or directly impact the underlying cause of the heart disease or disorder to be considered effective treatment.

As used herein, the term “short term,” when applied to treatment with amiodarone and ivabradine for engraftment arrhythmia, means treatment only so long as engraftment arrhythmia continues to occur. It is demonstrated herein that treatment with this combination of drugs reduces engraftment arrhythmia burden, and can be safely withdrawn after engraftment arrhythmia is resolved, without recrudescence of arrhythmia. Thus, while exact timing will likely vary for different subjects, in some embodiments short term treatment will be on the order of weeks (e.g., 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks or fewer) to months (e.g., 1 month, 2 months, 3 months, 4 months or fewer) post graft.

As used herein, “prevention” or “preventing,” when used in reference to a disease, disorder or symptoms thereof, refers to a reduction in the likelihood that an individual will develop a disease or disorder, e.g., heart failure following myocardial infarction, as but one example. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, pigs, guinea pigs, hamsters etc.). A subject can have previously received a treatment for a cardiovascular disease or cardiac event, or has never received treatment for a cardiovascular disease or a cardiac event. A subject can have previously been diagnosed with having a cardiovascular disease, or has never been diagnosed with a cardiovascular disease.

As used herein the term “human stem cell” refers to a human cell that can self-renew and differentiate to at least one cell type. The term “human stem cell” encompasses human stem cell lines, human-derived induced pluripotent stem (iPS) cells, human embryonic stem cells, human pluripotent cells, human multipotent stem cells, amniotic stem cells, placental stem cells, or human adult stem cells.

As used herein, “in vitro-differentiated cardiomyocytes” refers to cardiomyocytes that are generated in culture, typically via step-wise differentiation from a precursor cell such as a human embryonic stem cell, an induced pluripotent stem cell, an early mesoderm cell, a lateral plate mesoderm cell or a cardiac progenitor cell.

As used herein, the term “anti-arrhythmic” or “anti-arrhythmic agent” refers any agent (e.g., small molecule or pharmaceutical composition) that reduces the onset, frequency, and/or severity of a cardiac arrhythmia. Anti-arrhythmics can be used to treat irregular cardiac rhythm, to reduce heart rate in tachycardias, increase heart rate in bradycardias, or otherwise promote a normal sinus rhythm and prevent sudden cardiac death.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell.

The phrase “pharmaceutically acceptable” is employed herein to refer to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, a “reference level” refers to the level of a marker or parameter in a normal, otherwise unaffected cell population or tissue (e.g., a cell, tissue, or biological sample obtained from a healthy subject, or a biological sample obtained from the subject at a prior time point, e.g., cell, tissue, or a biological sample obtained from a patient prior to being diagnosed with a disease, or a biological sample that has not been contacted with an agent or composition disclosed herein).

As used herein, an “appropriate control” refers to an untreated, otherwise identical cell, subject, organism, or population (e.g., a cell, tissue, or biological sample that was not contacted by an agent or composition described herein) relative to a cell, tissue, biological sample, or population contacted or treated with a given treatment. For example, an appropriate control can be a subject or a tissue that has not been administered amiodarone and ivabradine.

The term “statistically significant” or “significantly” refers to statistical significance and generally means a two standard deviation (2SD) or greater difference.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean±1%.

Cardiovascular Diseases

A cardiovascular disease is a disease that affects the heart and/or circulatory system of a subject. Non-limiting examples of cardiac diseases include atherosclerotic heart disease, cardiomyopathy, cardiac arrhythmias, congenital heart disease, myocardial infarction, heart failure, cardiac hypertrophy, valvular stenosis, regurgitation, ischemia, fibrillation, and polymorphic ventricular tachycardia. Symptoms of cardiovascular disease can include but are not limited to syncope, fatigue, shortness of breath, chest pain, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia. Methods of diagnosing arrhythmias are known in the art.

The term, “cardiac event” refers to an incident of a myocardial injury, myocardial infarction, ventricular fibrillation, stenosis, arrhythmia, or the like.

Cardiac electrophysiological and contractile function is a tightly controlled process. When ion channel regulation or contractile function is disrupted in a cardiac cell or tissue, this can result in cardiac arrhythmias that can sometimes be deadly. Cardiac diseases remain a leading cause of death worldwide.

Human stem cell derived cardiomyocytes have emerged as a promising treatment for cardiovascular diseases and cardiac injuries sustained from myocardial infarction. However, the functional maturity of in vitro-differentiated cardiomyocytes in existing models is generally lacking and these cardiomyocytes can cause arrhythmias following engraftment.

In one aspect, described herein is a method of treating a cardiovascular disease. In another aspect, described herein is a method of treating or ameliorating an engraftment arrhythmia in a subject recipient of a cardiac graft of cardiomyocytes, the method comprises: administering to the subject an effective amount of amiodarone and an effective amount of ivabradine.

In some embodiments of any of the aspects, the subject has or is at risk for having a cardiovascular disease or a cardiac event.

In some embodiments of any of the aspects, the subject having a cardiovascular disease is in need of or has received a cardiac graft. In some embodiments, the subject has or is diagnosed with an engraftment arrhythmia. In some aspects, described herein is a method of preventing or reducing an engraftment arrhythmia.

An engraftment arrhythmia is a novel and aberrant cardiac rhythm that occurs following administration of a graft of cardiac cells or cardiomyocytes. Engraftment arrhythmias are observed after cardiac graft transplantation and generally persist transiently for days to weeks. Engraftment arrhythmia can cause sudden cardiac death and heart failure in the subject.

Methods of treating a cardiovascular diseases and arrhythmias are known in the art. One classic example of therapeutics used in the treatment of cardiovascular disease, specifically arrhythmias, includes anti-arrhythmic agents.

### Cardiomyocytes for Cardiac Engraftment

Cardiac engraftment administers cardiomyocytes to a site of cardiac injury in the heart. A skilled physician can determine the site of injury by methods known in the art. A primary goal of cardiac engraftment is to provide electrical and mechanical stability to the injured myocardium that cannot be achieved by pharmaceutical treatments alone.

The following describes various sources and stem cells that can be used to prepare cardiomyocytes for engraftment into a subject.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. Pluripotent stem cells can differentiate into cells derived from any of the three germ layers.

Cardiomyocytes useful in the compositions and methods described herein can be differentiated from both embryonic stem cells and induced pluripotent stem cells, among others. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells: Embryonic stem cells and methods for their retrieval are well known in the art and are described, for example, in Trounson A O Reprod Fertil Dev (2001) 13: 523, Roach M L Methods Mol Biol (2002) 185: 1, and Smith A G Annu Rev Cell Dev Biol (2001) 17:435. The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin. Induced Pluripotent Stem Cells (iPSCs):

In some embodiments, the compositions and methods described herein utilize cardiomyocytes that are differentiated in vitro from induced pluripotent stem cells. An advantage of using iPSCs to generate cardiomyocytes for the compositions described herein is that, if so desired, the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects. While this is an advantage of iPS cells, in alternative embodiments, the cardiomyocytes useful for the methods and compositions described herein are derived from non-autologous sources (e.g., allogenic). In addition, the use of iPSCs negates the need for cells obtained from an embryonic source. Thus, in one embodiment, the stem cells used to generate cardiomyocytes for use in the methods and compositions described herein are not embryonic stem cells.

Although differentiation is generally irreversible under physiological contexts, several methods have been developed in recent years to reprogram somatic cells to induced pluripotent stem cells. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell. It should be noted that placing many primary cells in culture can lead to some loss of fully differentiated characteristics. However, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. Thus, cells can be terminally differentiated somatic cells, as well as from adult stem cells, or somatic stem cells.

In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state with the capacity for self-renewal and differentiation to cells of all three germ layer lineages. These are induced pluripotent stem cells (iPSCs or iPS cells).

Methods of reprogramming somatic cells into iPS cells are known in the art. See for example, U.S. Pat. Nos. 8,129,187 B2; 8,058,065 B2; US Patent Application 2012/0021519 A 1; Singh et al. Front. Cell Dev Biol. (2015); and Park et al. Nature (2008); which are incorporated by reference in their entireties. Specifically, iPSCs are generated from somatic cells by introducing a combination of reprogramming transcription factors. The reprogramming factors can be e.g., nucleic acids, vectors, small molecules, viruses, polypeptides, or any combination thereof. Non-limiting examples of reprogramming factors include Oct4 (Octamer binding transcription factor-4), Sox2 (Sex determining region Y)-box 2, Klf4 (Kruppel Like Factor-4), and c-Myc. Factors (e.g., LIN28+Nanog, Esrrb, Pax5 shRNA, C/EBPa, p53 siRNA, UTF1, DNMT shRNA, Wnt3a, SV40 LT(T), hTERT) or chemicals (e.g., BIX-01294, BayK8644, RG108, AZA, dexamethasone, VPA, TSA, SAHA, PD025901+CHIR99021(2i), A-83-01) have been found to replace one or the other reprogramming factors from basal reprogramming factors or to enhance the efficiency of reprogramming.

The specific approach or method used to generate pluripotent stem cells from somatic cells (e.g., any cell of the body with the exclusion of a germ line cell; fibroblasts, etc.) is not critical to the claimed invention. Thus, any method that re-programs a somatic cell to the pluripotent phenotype would be appropriate for use in the methods described herein.

The efficiency of reprogramming (i.e., the number of reprogrammed cells) derived from a population of starting cells can be enhanced by the addition of various small molecules as shown by Shi, Y., et al. (2008) Cell-Stem Cell 2:525-528, Huangfu, D., et al. (2008) Nature Biotechnology 26(7):795-797, and Marson, A., et al. (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of one or more stem cell markers. Such expression in a cell derived from a somatic cell identifies the cells as induced pluripotent stem cells. Stem cell markers can include but are not limited to SSEA3, SSEA4, CD9, Nanog, Oct4, Fbx15, Ecatl, Esg 1, Eras, Gdf3, Fgf4, Cripto, Daxl, Zpf296, Slc2a3, Rexl, Utfl, and Natl, among others. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

Adult Stem Cells: Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns. It is contemplated that cardiomyocytes differentiated from adult stem cells can also be used for cardiac grafts as described herein. Methods of isolating adult stem cell are known in the art. See for example, U.S. Pat. No. 9,206,393 B2; and US Application No. 2010/0166714 A1; which are incorporated herein by reference in their entireties.

### In Vitro-Differentiation

The methods and compositions described herein use in vitro-differentiated cardiomyocytes. Methods for the differentiation of either cell type from ESCs or iPSCs are known in the art. See, e.g., LaFlamme et al., Nature Biotech 25:1015-1024 (2007), which describes the differentiation of cardiomyocytes which is incorporated herein by reference in its entirety.

In certain embodiments, the step-wise differentiation of ESCs or iPSCs to cardiomyocytes proceeds in the following order: ESC or iPSC >cardiogenic mesoderm >cardiac progenitor cells >cardiomyocytes (see e.g., Lian et al. Nat Prot (2013); US Applicant No. 2017/0058263 A1; 2008/0089874 A1; 2006/0040389 A1; U.S. Pat. Nos. 10,155,927 B2; 9,994,812 B2; and 9,663,764 B2, the contents of each of which are incorporated herein by reference their entireties). A number of protocols for differentiating ESCs and iPSCs to cardiomyocytes are known in the art. For example, agents can be added or removed from cell culture media to direct differentiation to cardiomyocytes in a step-wise fashion. Non-limiting examples of factors and agents that can promote cardiomyocyte differentiation include small molecules (e.g., Wnt inhibitors, GSK3 inhibitors), polypeptides (e.g., growth factors), nucleic acids, vectors, and patterned substrates (e.g., nanopatterns). The addition of growth factors necessary in cardiovascular development, including but not limited to fibroblast growth factor 2 (FGF2), transforming growth factor β (TGFβ) superfamily growth factors-Activin A and BMP4, vascular endothelial growth factor (VEGF), and the Wnt inhibitor DKK-1, can also be beneficial in directing differentiation along the cardiac lineage. Additional examples of factors and conditions that help promote cardiomyocyte differentiation include but are not limited to B27 supplement lacking insulin, cell-conditioned media, external electrical pacing, and nanopatterned substrates, among others.

Cardiac/Cardiomyocyte grafts

In one aspect, described herein is a method of cardiomyocyte graft or transplant, the method comprises: a) administering cardiomyocytes to cardiac tissue of a subject in need thereof; and b) administering to the subject an amount of amiodarone and an amount of ivabradine effective to reduce engraftment arrhythmia in the subject.

As used herein, the term “transplanting” or “transplant” is used in the context of the placement of cells, e.g. cardiomyocytes, as described herein into a subject, by a method or route which results in at least partial localization of the introduced cells at a desired site, such as a site of injury or repair, such that a desired effect(s) is produced. The cells e.g. cardiomyocytes, or their differentiated progeny (e.g. cardiac fibroblasts etc.) and cardiomyocytes can be implanted directly or into the cardiac tissue of the recipient, e.g., at or near a site, or into cardiac tissue of a subject with a cardiac disease. As one of skill in the art will appreciate, long-term engraftment of the cardiomyocytes is desired as cardiomyocytes generally do not proliferate to an extent that the heart can heal from an acute injury comprising cell death. In some embodiments, the cells are optionally transplanted on or within a scaffold or biocompatible material that supports viability of the implanted cardiomyocytes, and/or, for example, assists with keeping administered cells in the desired location for engraftment or promotes integration with native cardiac cells in a subject. Preferably, the cardiomyocytes are human stem cell derived-cardiomyocytes or in vitro-differentiated cardiomyocytes as described herein. In some embodiments, the in vitro-differentiated cardiomyocytes are differentiated in vitro from embryonic stem cells or from iPS cells.

A scaffold is a structure, comprising a biocompatible material including but not limited to a gel, sheet, or lattice that can contain the cells in a desired location but permit the entry or diffusion of factors in the environment necessary for survival and function. A number of biocompatible polymers suitable for a scaffold are known in the art.

One of skill in the art can determine the number of cardiomyocytes needed for a graft. In some embodiments, about 10 million cardiomyocytes to about 10 billion cardiomyocytes are administered to the subject. For use in the various aspects described herein, an effective amount of human cardiomyocytes can comprise at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹, at least 6×10⁹, at least 7×10⁹, at least 8×10⁹, at least 9×10⁹, at least 1×10¹⁰, or more cardiomyocytes for engraftment.

To reduce the onset and severity of an engraftment arrhythmia in a subject receiving the cardiac graft cardiomyocytes, the methods described herein further comprise administering to the subject an effective amount of amiodarone and an effective amount of ivabradine. The combination of these anti-arrhythmic agents is demonstrated herein in the working examples to reduce and treat engraftment arrhythmia.

Amiodarone is a class III antiarrhythmic agent prescribed for the treatment of cardiac arrest, ventricular tachycardia, and atrial fibrillation. Analogs and derivatives of amiodarone are known in the art, see e.g., US Patent Nos. 7,799,799 B2; 9,018,250 B2; and Carlsson et al. J Med Chem. 2002 Jan 31;45(3):623-30, which are incorporated herein by reference in their entireties. It is contemplated that amiodarone analogs and derivatives can also be beneficial for treating engraftment arrhythmias.

The mechanism of action of amiodarone is that the drug inhibits voltage-gated potassium channels and voltage-gated calcium channels, which in turn prolongs phase 3 of the cardiac action potential. Specifically, amiodarone inhibits the pore-forming subunit of the potassium ion channel, Kᵥ11.1 (encoded by the KCNH2 gene) and inhibits the voltage-gated calcium channel (encoded by the CACNA2D2 gene). However, amiodarone has also been shown to inhibit voltage-gated sodium channel activity, which can contribute to the drug's pro-arrhythmic potential.

Administration of amiodarone exhibits beta-blocker like activity, in that it reduces heart rate when administered to a subject. The clinical effects of amiodarone include the prolongation of the QT interval due to the increased refractory periods of the ventricles, bundles of His, and Purkinje fibers. While this effect is beneficial for the treatment of arrhythmias such as atrial fibrillation, this effect can become pro-arrhythmic. It is well known in the art that amiodarone and other anti-arrhythmic agents can lead to drug-induced QT prolongation depending on the dose and treatment regimen. This can be exacerbated when combined with other drugs or anti-arrhythmic agents.

Ivabradine is a class I_{f} anti-arrhythmic agent that is used for the treatment of angina, tachycardia, and heart failure. Analogs and derivatives of ivabradine are known in the art. See for example, U.S. Pat. Nos. 7,879,842; 7,361,650; 7,867,996; and 7,361,649, which are incorporated herein by reference in their entireties. It is contemplated that certain ivabradine analogs and derivatives can also be beneficial for the methods described herein.

Ivabradine is known to inhibit funny channels (also known as HCN channels) in the heart. There are 4 isoforms of HCN channels encoded by the HCN genes, HCN1-HCN 4. The major function of funny current (If) in the heart is to maintain the pacemaker activity in the SA node. Blocking funny channels with ivabradine results in an overall reduction in heart rate.

The combination of amiodarone with ivabradine as described herein reduces the rate and burden of arrhythmia in subjects with cardiomyocyte grafts. This combination of anti-arrhythmic agents suggested improvements in cardiovascular survival of subjects, as shown in the working examples.

### Administration and Efficacy

In one aspect, described herein are methods for treating or ameliorating a cardiac disease, disorder, event, or injury comprising administering cardiomyocytes to a subject in need thereof and administering an effective amount of amiodarone and ivabradine. In some embodiments, methods described herein prevent an anticipated disorder e.g., an engraftment arrhythmia.

In another aspect, described herein are methods for treating or ameliorating an engraftment arrhythmia.

The anti-arrhythmic agent described herein can be administered by any appropriate route which results in a reduction in arrhythmic burden in the subject. In some embodiments of any of the aspects, the term “administering” refers to the administration of a pharmaceutical composition comprising one or more agents. The administering can be done by direct injection (e.g., directly administered to a target cell or tissue), subcutaneous injection, intramuscular injection, oral, or nasal delivery, or a combination thereof to the subject in need thereof.

As discussed above, the cells are administered directly to the cardiac tissue. Anti-arrhythmic agents, e.g., amiodarone and ivabradine, can be administered in any suitable manner including but not limited to oral, parenteral, intravenous (IV), intramuscular, subcutaneous, transdermal, airway (aerosol), pulmonary, cutaneous, topical, or injection administration. Administration can be local or systemic.

Administration of amiodarone and ivabradine can be IV or oral for both drugs. Thus, it is contemplated that both drugs (or analog thereof) can be administered orally, both IV, or one orally and the other IV in either combination. However, for ease of administration and patient compliance, it is preferred that both drugs are administered orally.

An “effective amount” as used herein refers to the amount of amiodarone and/or ivabradine or analog thereof needed to alleviate an engraftment arrhythmia. By preventing or alleviating engraftment arrhythmia, engraftment and integration of grafted cells can be facilitated and the clinical outcome for the subject can be improved, including reduced risk for heart failure or sudden cardiac death. By “alleviate” in this context is meant a reduction in arrhythmia burden of at least 10% relative to arrhythmia occurring or expected to occur without administration of amiodarone and ivabradine as described herein. Arrhythmic burden can be calculated as described in the working examples herein or as known in the art. Arrhythmic burden without the drug regimen described can be 75% or more. This can be reduced by at least 10% with the administration of amiodarone and ivabradine. It is understood that for any given case, an appropriate “effective amount” can be determined by one of ordinary skill in the art using only routine experimentation.

In some embodiments of any of the aspects, the administration of amiodarone and ivabradine or analog thereof reduces post-graft accelerated resting heart rate experienced by the graft recipient by at least 10% or more, 20% or more, 30% or more, 40% or more, 50% or more, 60% or more relative to a subject receiving a graft of the same type of cells in the absence of amiodarone and ivabradine administration or analog thereof.

Thus, in some embodiments of any of the aspects, the administration of amiodarone and ivabradine or analog thereof reduces engraftment arrhythmia burden, i.e., the proportion of time in which the subject experiences engraftment arrhythmia, by at least 10%, at least 20%, at least 30%, at least 40%, at least 50%, at least 60%, at least 70%, at least 80%, at least 90%, at least 99% or more relative to a subject receiving a graft of the same type of cardiomyocytes in the absence of amiodarone and ivabradine administration. While at least 10% reduction is considered effective treatment, it is contemplated that administration of amiodarone and ivabradine permit up to complete cessation of engraftment arrhythmia.

The effective dose can be estimated initially from cell culture assays, and a dose range can be formulated in animals (e.g., pig). Data obtained from cell culture assays and animal studies can be used in formulating a range of dosage for use in humans. The dosage of such anti-arrhythmic agents lies preferably within a range of circulating concentrations that include the ED₅₀ with little or no toxicity. The dosage may vary within this range depending upon the dosage form employed and the route of use or administration utilized.

Generally, the compositions are administered so that the amiodarone is used or given at a dose from at least 50 mg, at least 100 mg, at least 150 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, to about 1000 mg.

In some embodiments of any of the aspects, the amiodarone or analog thereof is administered orally at a dose of 100-800 mg, three times per day. In some embodiments of any of the aspects, the amiodarone is administered by IV bolus at a dose of 100-300 mg. In some embodiments of any of the aspects, the amiodarone is administered to a serum concentration of 1.5 to 2.5 μg/ml.

In some embodiments, ivabradine administered at a dose from at least 1 mg, at least 2 mg, at least 3 mg, at least 4 mg, at least 5 mg, at least 6 mg, at least 7 mg, at least 8 mg, at least 9 mg, at least 10 mg, at least 11 mg, at least 12 mg, at least 13 mg, at least 14 mg, at least 15 mg, at least 16 mg, at least 17 mg, at least 18 mg, at least 19 mg, to about 20 mg.

In some embodiments of any of the aspects, the ivabradine or analog thereof is orally administered at a dose of 5 to 15 mg, twice per day.

In some embodiments, the administration of amiodarone is a single bolus administration. In some embodiments, the administration is continuous or repeated administration. In some embodiments, the administration is oral administration and/or intravenous injection. In some embodiments, the amiodarone is administered orally at a dose of 100-800 mg, three times per day. In some embodiments, the amiodarone is administered by IV bolus at a dose of 100-300 mg. In some embodiments, the amiodarone is administered to a serum concentration of 1.5 to 2.5 μg/ml. In some embodiments, the ivabradine is orally administered at a dose of 5 to 15 mg, twice per day. In some embodiments, the ivabradine is administered when there is tachycardia. In some embodiments, the ivabradine is administered to maintain a resting heart rate of less than or equal to 150 beats per minute (bpm). In some embodiments, the administration of amiodarone and ivabradine is short-term. In some embodiments, the administration of amiodarone and ivabradine is terminated after engraftment arrhythmia burden reaches zero, without recrudescence of the arrhythmia.

The anti-arrhythmic agents, amiodarone and ivabradine or analog thereof described herein, are used in combination for treating an engraftment arrhythmia and/or a cardiovascular disease. Administered “in combination,” as used herein, means that two (or more) different treatments are delivered to the subject during the course of the subject's affliction with the disorder, e.g., the two or more treatments are delivered after the subject has been diagnosed with the disorder (a cardiovascular disease or an engraftment arrhythmia) and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The anti-arrhythmic agents described herein and/or at least one additional therapy can be administered simultaneously, in the same or in separate compositions, or sequentially. For sequential administration, the amiodarone described herein can be administered first, and the ivabradine can be administered second, or the order of administration can be reversed. The agent and/or other therapeutic agents, procedures or modalities can be administered during periods of active disorder (e.g., during an engraftment arrhythmia), or during a period of remission or less active disease (e.g., before or after engraftment). The anti-arrhythmic agents can be administered before cardiac grafting, concurrently with the treatment, post-treatment, or during a flare of a cardiovascular disease following engraftment.

When administered in combination, the amiodarone and the ivabradine can be administered in an amount or dose that is higher, lower, or the same as the amount or dosage of each anti-arrhythmic agent used individually, e.g., as a monotherapy. In certain embodiments, the administered amount or dosage of the amiodarone, ivabradine, or both, is lower (e.g., at least 20%, at least 30%, at least 40%, or at least 50%) than the amount or dosage of each anti-arrhythmic agent used individually. In other embodiments, the amount or dosage of amiodarone, ivabradine, or both, that results in a desired effect (e.g., treatment of a cardiovascular disease) is lower (e.g., at least 20%, at least 30%, at least 40%, or at least 50% lower) than the amount or dosage of each agent individually required to achieve the same therapeutic effect.

In some embodiments of any of the aspects, the amiodarone and ivabradine or an analog thereof are administered concurrently with the graft of cardiomyocytes. In some embodiments of any of the aspects, administration of amiodarone is initiated prior to administration of the graft of cardiomyocytes. In some embodiments of any of the aspects, administration of ivabradine is initiated prior to administration of the graft of cardiomyocytes. In some embodiments of any of the aspects, administration of both amiodarone and ivabradine is initiated prior to administration of the graft of cardiomyocytes. In some embodiments of any of the aspects, administration of ivabradine is initiated concurrently with or after administration of the graft of cardiomyocytes. In some embodiments of any of the aspects, administration of amiodarone is initiated concurrently with or after administration of the graft of cardiomyocytes.

In some embodiments of any of the aspects, the amiodarone is administered beginning at least 1 hour, at least 5 hours, at least 10 hours, at least 15 hours, at least 20 hours, least 1 day, at least 2 days, at least 3 days, at least 4 days, at least 5 days, at least 6 days, at least 7 days, at least 8 days, at least 9 days, at least 10 days, prior to engraftment. In some embodiments of any of the aspects, the ivabradine is administered beginning at least 1 hour, at least 5 hours, at least 10 hours, at least 15 hours, at least 20 hours, least 1 day, at least 2 days, at least 3 days, at least 4 days, at least 5 days, at least 6 days, at least 7 days, at least 8 days, at least 9 days, at least 10 days, prior to engraftment.

In some embodiments, of any of the aspects, ivabradine is administered as needed to control heart rate, i.e., to limit or control tachycardia as commonly occurs with engraftment arrhythmia. In such embodiments, amiodarone is administered either before (e.g., beginning 7 days, 6 days, 5 days, 4 days, 3 days, 2 days, 1 day) or concurrently with graft administration and continues after grafting.

In some embodiments of any of the aspects, the amiodarone is administered at least once per day, twice per day, three times per day, or more. In some embodiments of any of the aspects, the ivabradine is administered at least once per day, twice per day, three times per day, or more.

In additional embodiments, other types of anti-arrhythmics can be administered concurrently or in addition to amiodarone and ivabradine to aid in treatment of the subject.

In certain embodiments, amiodarone and/or ivabradine or analogs thereof can be administered as needed for rate control in the subject and to alleviate at least one symptom of an engraftment arrhythmia. The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen.

In some embodiments of any of the aspects, the methods described herein comprise administering an effective amount of amiodarone and ivabradine to a subject in order to alleviate at least one symptom of the engraftment arrhythmia and/or cardiovascular disease. As used herein, “alleviating at least one symptom of the cardiovascular disease” or “alleviating at least one symptom of an engraftment arrhythmia” is ameliorating any condition or symptom associated with the cardiovascular disease (e.g., fatigue, shortness of breath, syncope, chest pain) and includes, for example, reduction of the arrhythmia itself. As compared with an equivalent untreated control, such reduction is by at least 10%, 20%, 30%, 40%, 50%, 60%, 70%, 80%, 90%, 95%, 99% or more as measured by any standard technique.

In some embodiments of any of the aspects, after an initial treatment regimen, the treatments can be administered on a less frequent basis. For example, administration daily for three weeks, administration can be reduced to every other day, every three days, weekly, or less frequently as warranted by the subject's incidence of arrhythmia. Alternatively, dosing can remain as frequent but be reduced in amount(s).

In some embodiments of any of the aspects, the subject is first diagnosed as having a cardiovascular disease or disorder prior to administering a cardiomyocyte graft as described herein. In some embodiments, the subject is first diagnosed as being at risk of developing a cardiac disease (e.g., myocardial injury) or disorder prior to administering the cells.

In some embodiments, the ivabradine or an analog thereof is administered when there is tachycardia. Adult human resting heart rate is generally 60 to 100 beats per minute (bpm). Tachycardia is a resting heart rate greater than this. However, while a resting heart rate of 60 to 100 bpm is a goal, patients can typically manage with a rate of less than 150 bpms. Thus, ivabradine can be administered for tachycardia after grafting with a goal of maintaining resting heart rate between 100 and 150 bpms, preferably less than 140 bpm, less than 130bpm, less than 120 bpm, or less than 110 bpm

Unless otherwise explained, all technical and scientific terms used herein have the same meaning as commonly understood by one of ordinary skill in the art to which this disclosure belongs.

It should be understood that this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

Some embodiments of the technology described herein can be defined according to any of the following numbered paragraphs:
1. A method of treating or ameliorating an engraftment arrhythmia in a subject recipient of a cardiac graft of cardiomyocytes, the method comprising administering to the subject an effective amount of amiodarone and an effective amount of ivabradine.
2. The method of paragraph 1, wherein the cardiac graft of cardiomyocytes comprises in vitro-differentiated cardiomyocytes.
3. The method of paragraph 2, wherein the in vitro-differentiated cardiomyocytes are differentiated from induced pluripotent stem (iPS) cells or from embryonic stem (ES) cells.
4. The method of any of paragraphs 1-3, wherein the cardiac graft of cardiomyocytes is derived from stem cells autologous to the subject.
5. The method of any of paragraphs 1-3, wherein the cardiac graft of cardiomyocytes is derived from stem cells allogeneic to the subject.
6. The method of any one of paragraphs 1-5, wherein the amiodarone and ivabradine are administered concurrently with the cardiac graft of cardiomyocytes.
7. The method of any one of paragraphs 1-5, wherein administration of amiodarone is initiated prior to administration of the cardiac graft of cardiomyocytes.
8. The method of any one of paragraphs 1-5, wherein administration of ivabradine is initiated prior to administration of the cardiac graft of cardiomyocytes.
9. The method of any one of paragraphs 1-5, wherein administration of both amiodarone and ivabradine is initiated prior to administration of the cardiac graft of cardiomyocytes.
10. The method of any one of paragraphs 1-5, wherein the administration of ivabradine is initiated concurrently with or after administration of the cardiac graft of cardiomyocytes.
11. The method of any one of paragraphs 1-5, wherein the administration of amiodarone is initiated concurrently with or after administration of the cardiac graft of cardiomyocytes.
12. The method of any one of paragraphs 1-11, wherein the administration of amiodarone is a single bolus administration.
13. The method of any one of paragraphs 1-11, wherein the administration is continuous or repeated administration.
14. The method of any one of paragraphs 1-13, wherein the administration is oral administration and/or intravenous injection.
15. The method of any one of paragraphs 1-14, wherein the amiodarone is administered orally at a dose of 100-800 mg, three times per day.
16. The method of any one of paragraphs 1-14, wherein the amiodarone is administered by IV bolus at a dose of 100-300 mg.
17. The method of any one of paragraphs 1-15, wherein the amiodarone is administered to a serum concentration of 1.5 to 2.5 μg/ml.
18. The method of any one of paragraphs 1-17, wherein the ivabradine is orally administered at a dose of 5 to 15 mg, twice per day.
19. The method of any one of paragraphs 1-18, wherein the ivabradine is administered when there is tachycardia.
20. The method of any one of paragraphs 1-19, wherein ivabradine is administered to maintain a resting heart rate of less than or equal to 150 beats per minute (bpm).
21. The method of any one of paragraphs 1-20, wherein the administration of amiodarone and ivabradine reduces post-graft accelerated heart rate experienced by the graft recipient by at least 10% relative to a subject receiving a graft of the same type of cells in the absence of amiodarone and ivabradine administration.
22. The method of any one of paragraphs 1-21, wherein the administration of amiodarone and ivabradine reduces the proportion of time in which the subject experiences engraftment arrhythmia by at least 10% relative to a subject receiving a graft of the same type of cardiomyocytes in the absence of amiodarone and ivabradine administration.
23. The method of any one of paragraphs 1-22, wherein administration of amiodarone and ivabradine is short-term.
24. The method of any one of paragraphs 1-22, wherein administration of amiodarone and ivabradine is terminated after engraftment arrhythmia burden reaches zero, without recrudescence of the arrhythmia.
25. A method of cardiomyocyte transplant, the method comprising:
a) administering in vitro-differentiated cardiomyocytes to cardiac tissue of a subject in need thereof;
b) administering to the subject an amount of amiodarone and an amount of ivabradine effective to reduce engraftment arrhythmia in the subject.
26. The method of paragraph 25, wherein engraftment arrhythmia is reduced in the subject relative to a subject receiving in vitro-differentiated cardiomyocytes without receiving amiodarone and ivabradine.
27. The method of any one of paragraphs 25 or 26, wherein the in vitro-differentiated cardiomyocytes are differentiated in vitro from embryonic stem cells or from iPS cells.
28. The method of paragraph 27, wherein the iPS cells are autologous to the subject.
29. The method of paragraph 27, wherein the iPS cells are allogeneic to the subject.
30. The method of any one of paragraphs 25-29, wherein the amiodarone and ivabradine are administered concurrently with the in vitro-differentiated cardiomyocytes.
31. The method of any one of paragraphs 25-29, wherein administration of amiodarone is initiated prior to administration of the in vitro-differentiated cardiomyocytes.
32. The method of any one of paragraphs 25-29, wherein administration of ivabradine is initiated prior to administration of the in vitro-differentiated cardiomyocytes.
33. The method of any one of paragraphs 25-29, wherein administration of both amiodarone and ivabradine is initiated prior to administration of the in vitro-differentiated cardiomyocytes.
34. The method of any one of paragraphs 25-29, wherein the administration of ivabradine is initiated concurrently with or after administration of the in vitro-differentiated cardiomyocytes.
35. The method of any one of paragraphs 25-29, wherein the administration of amiodarone is initiated concurrently with or after administration of the in vitro-differentiated cardiomyocytes.
36. The method of any one of paragraphs 25-35, wherein the administration of ivabradine is a single bolus administration.
37. The method of any one of paragraphs 25-36, wherein the administration is continuous or repeated administration.
38. The method of any one of paragraphs 25-37, wherein the administration is oral administration and/or intravenous (IV) injection.
39. The method of any one of paragraphs 25-38, wherein the amiodarone is administered orally at a dose of 100-800 mg, three times per day.
40. The method of any one of paragraphs 25-39, wherein the amiodarone is administered by IV bolus at a dose of 100-300 mg.
41. The method of any one of paragraphs 25-40, wherein the amiodarone is administered to a serum concentration of 1.5 to 2.5 μg/ml.
42. The method of any one of paragraphs 25-41, wherein the ivabradine is orally administered at a dose of 5 to 15 mg, twice per day.
43. The method of any one of paragraphs 25-42, wherein the ivabradine is administered when there is tachycardia.
44. The method of any one of paragraphs 25-43, wherein ivabradine is administered to maintain a resting heart rate of less than or equal to 150 beats per minute (bpm).
45. The method of any one of paragraphs 25-44, wherein administration of amiodarone and ivabradine is short-term.
46. The method of any one of paragraphs 25-45, wherein administration of amiodarone and ivabradine is terminated after engraftment arrhythmia burden reaches zero, without recrudescence of the arrhythmia.
47. The method of any one of paragraphs 1-46, wherein about 10 million cardiomyocytes to about 10 billion cardiomyocytes are administered to the subject.
48. The method of any one of paragraphs 1-47, wherein the subject is a human.
49. The method of any one of paragraphs 1-48, wherein the subject has or is at risk for having a cardiovascular disease or a cardiac event.
50. The method of paragraph 49, wherein the cardiovascular disease or the cardiac event is selected from the group consisting of: atherosclerotic heart disease, myocardial infarction, cardiomyopathy, cardiac arrhythmia, valvular stenosis, congenital heart disease, chronic heart failure, regurgitation, ischemia, fibrillation, and polymorphic ventricular tachycardia.
51. A composition comprising in vitro-differentiated cardiomyocytes, amiodarone and ivabradine.

### EXAMPLES

### Example 1: Amiodarone/Ivabradine Therapy Reduces Graft-Associated Arrhythmias

Cardiomyocyte replacement therapy is an area of active investigation, and can restore heart function after myocardial infarction. Human pluripotent stem cells (hPSC) can serve as a starting material for producing human cardiomyocytes in vitro. Large animal models of myocardial infarction have demonstrated the restorative potential for this candidate therapeutic (Chong et al., 2014; Shiba et al., 2016; Liu et al., 2018). However, hPSC-derived cardiomyocytes are electrophysiologically immature, and elicit cardiac arrhythmias in large animal models (ibid., Romagnuolo et al., 2019). These graft-induced arrhythmias appear shortly after the hPSC-derived cardiomyocytes are transplanted, and persist transiently for 3-4 weeks, during which the recipient is at risk for sudden cardiac death and heart failure. The observed return to normal sinus rhythm is hypothesized to reflect in vivo maturation of the transplanted hPSC-derived cardiomyocytes and suggests complete electrical integration with the host myocardium.

Reducing these transient graft-associated arrhythmias can significantly improve the safety profile of this therapeutic in myocardial infarct patients. While there are a number of anti-arrhythmic drugs available to heart disease patients, predicting their efficacy in cardiomyocyte replacement therapy is difficult because transplantation of immature cardiomyocytes is unlike any naturally occurring heart disease pathology or prior therapy. As a result, identifying one or more anti-arrhythmic drugs that are effective for this purpose needs to be empirically determined. This application describes a combination of two drugs that reduced the accelerated heart rate and arrhythmia burden caused by hPSC-derived cardiomyocyte transplants in pigs.

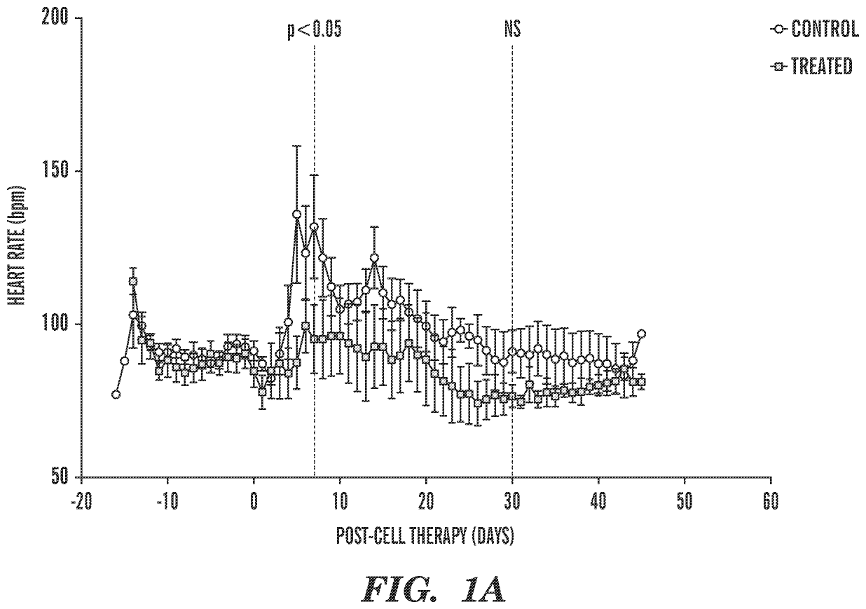

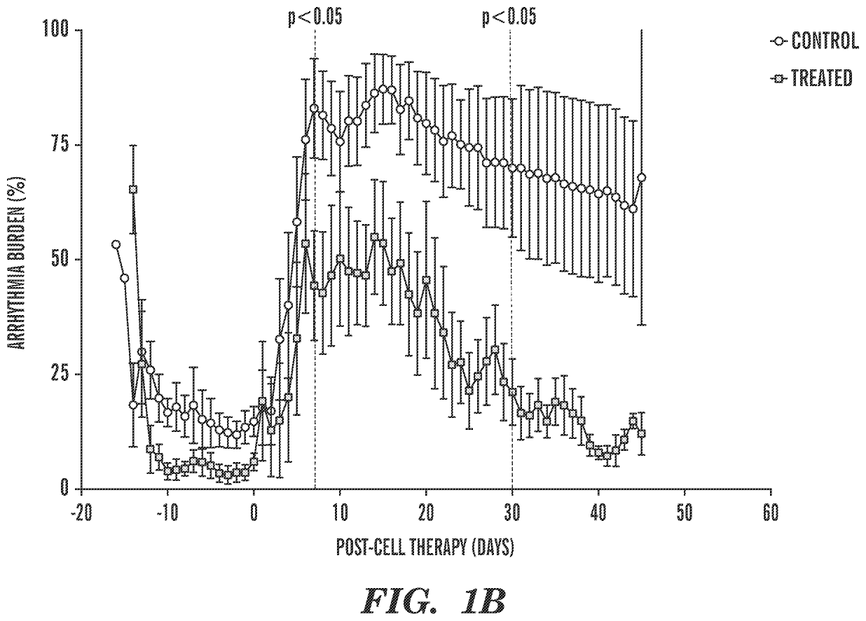

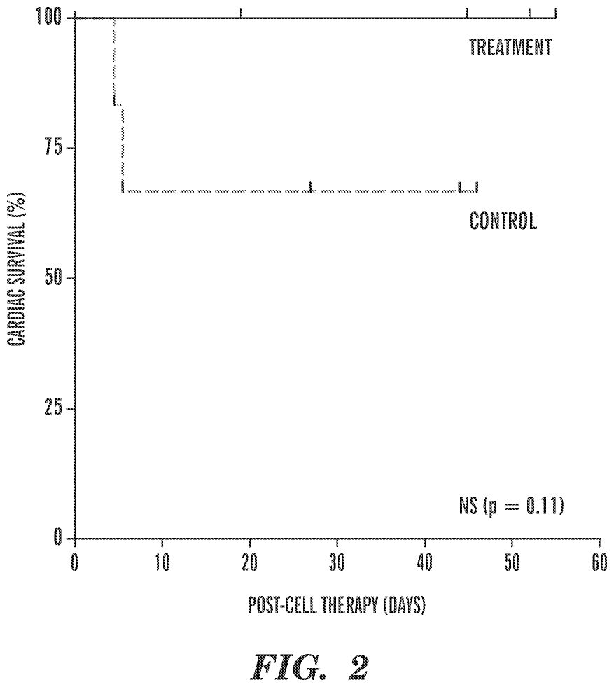

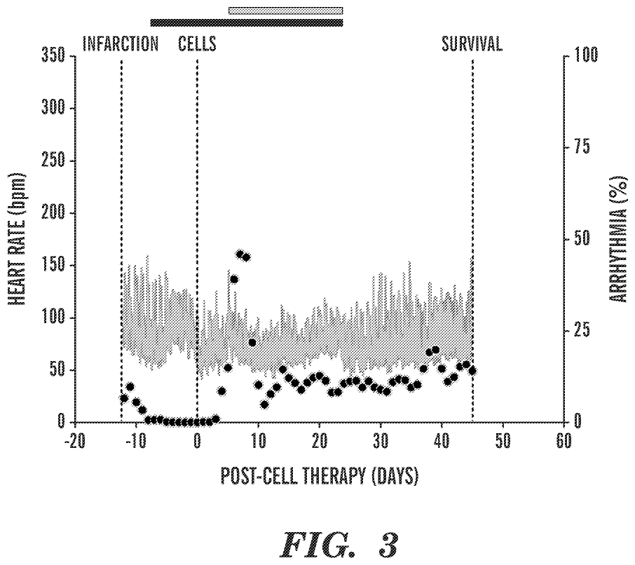

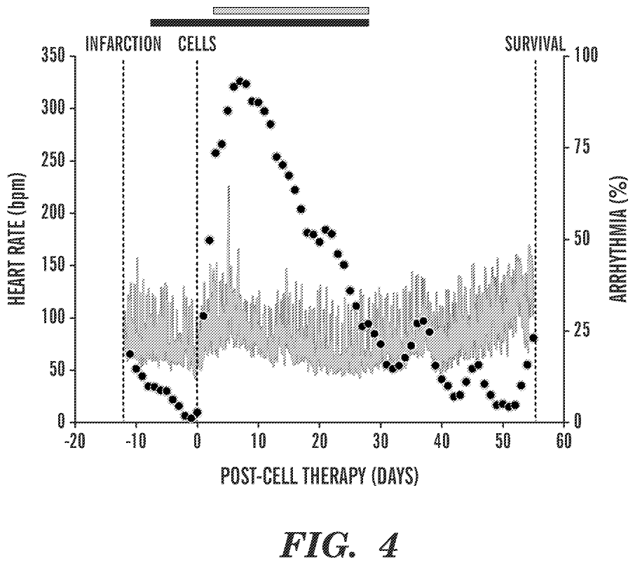

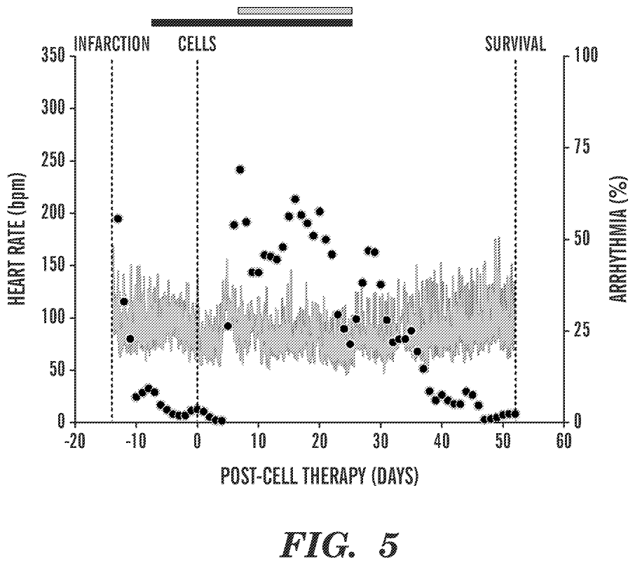

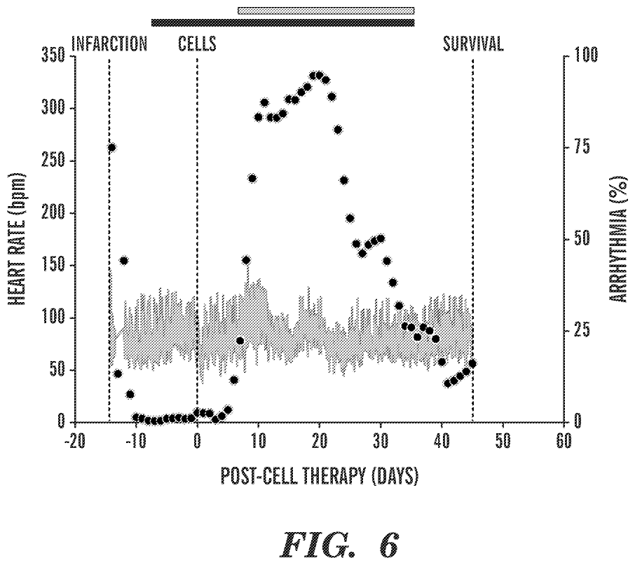

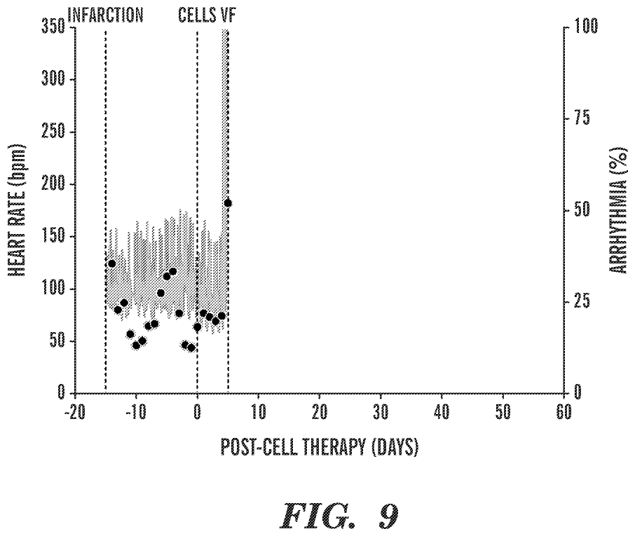

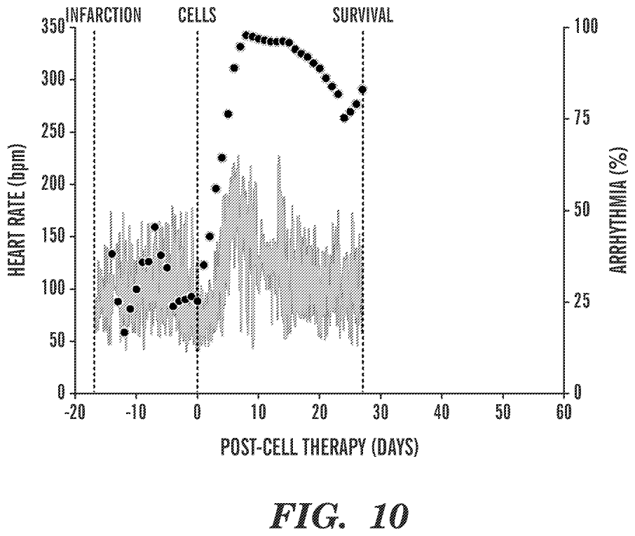

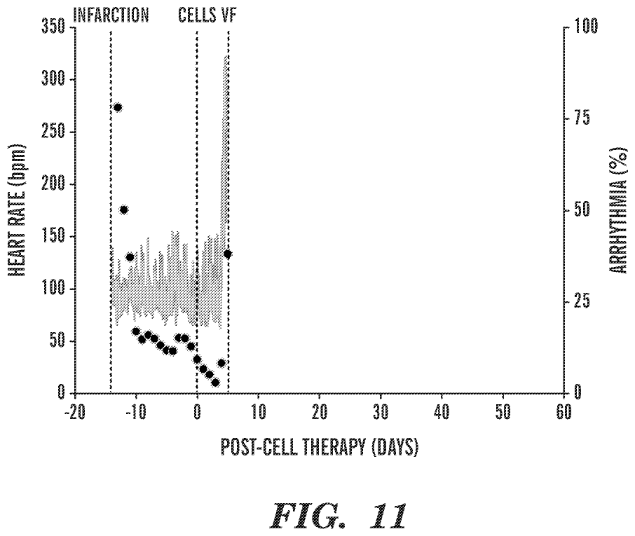

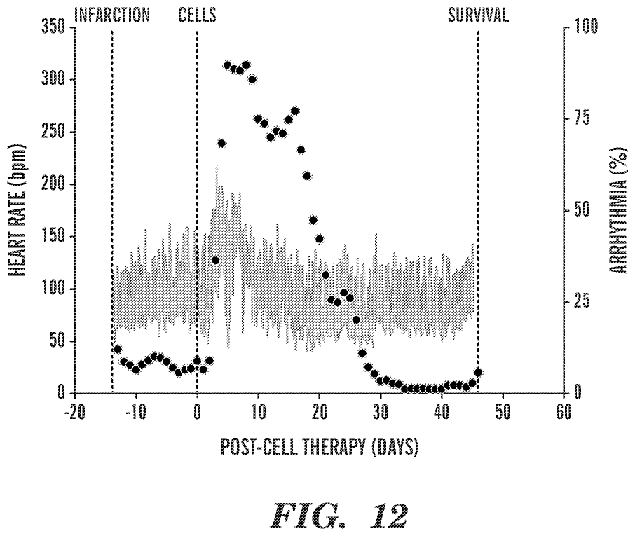

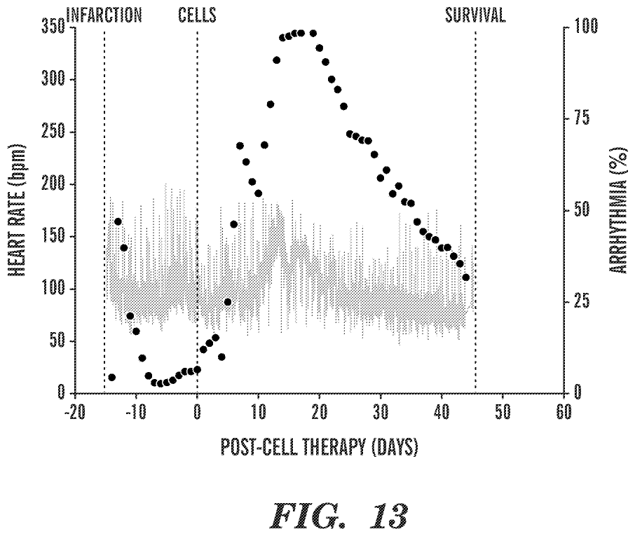

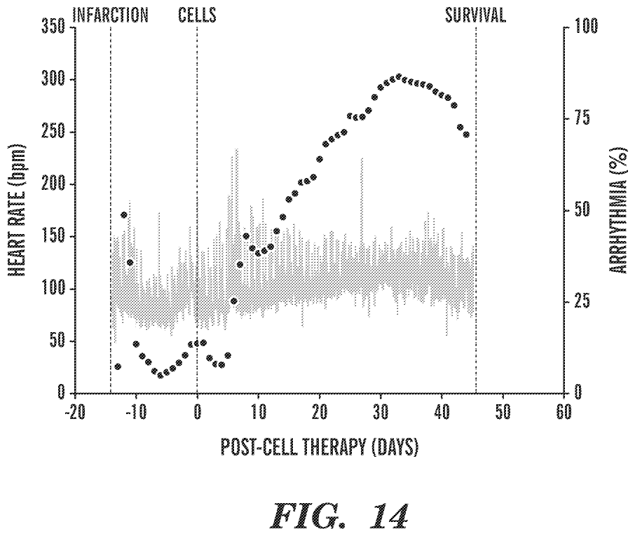

Experimental Design: Yucatan miniature pigs weighing 30-40 kg were used to model myocardial infarction. For this purpose, animals were subjected to a 90-minute occlusion of the left anterior descending coronary artery using a percutaneous transluminal coronary angioplasty balloon. An implanted electrocardiography device allowed continuous remote monitoring of the heart for the duration of the study. The hPSC-derived cardiomyocytes were administered two weeks after infarction, and the pigs were immunosuppressed to prevent immune rejection of the transplanted cells. In total, 500 million cardiomyocytes were delivered via percutaneous transendocardial injections. By design, the post-transplant study duration was at least 30 days. The control group (n=6) did not receive any anti-arrhythmic drugs. The treatment group (n=6), received a combination anti-arrhythmic drug therapy consisting of loading and maintenance dosing of amiodarone for target serum levels of 1.5 and 2.5 μg/ml and 5-15 mg twice a day of oral ivabradine. Novel Finding: The use of amiodarone/ivabradine therapy was found to reduce accelerated heart rate and arrhythmia burden in the treated pigs. This is reflected in the following pooled data for treated and control animals over the course of the study (FIGS. 1A-1B). The most surprising result was the impact of the amiodarone/ivabradine combinations on mortality (FIG. 2). In the absence of amiodarone/ivabradine therapy, two of the six control pigs died of ventricular fibrillation (FIG. 9, FIG. 11) and four survived (FIG. 10, FIG. 12-14). In contrast, none of the six pigs treated with amiodarone and ivabradine died of cardiovascular complications (FIGS. 3-8).

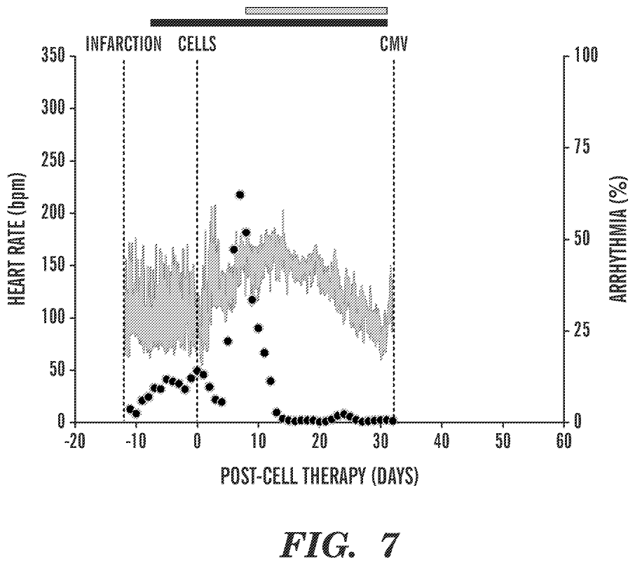

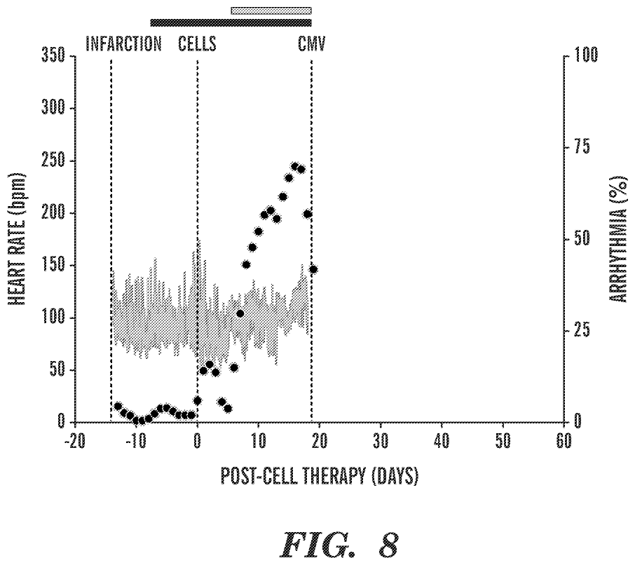

Results: Data for individual animals in this study are provided herein (FIGS. 1A-14). Note that the last two animals in the treatment group (amiodarone ±ivabradine) were euthanized due to complications related to immunosuppression (CMV infection reactivation) and was not cardiac in etiology (FIGS. 7-8).

In summary, the results provided herein show that amiodarone and ivabradine therapy reduces heart rate and arrhythmia burden in animals that received cardiac grafts compared with untreated animals (FIGS. 1A-1B) and the combination of these anti-arrhythmic agents improved survival of animals that received cell replacement therapy (FIG. 2).

### REFERENCES

Chong, J J, Yang X, Don C W, Minami E, Liu Y W, Weyers J J, Mahoney W M, Van Biber B, Palpant N J, Gantz J A, Fugate J A, Muskheli V, Gough G M, Vogel K W, Astley C A, Hotchkiss C E, Baldessari A, Pabon L, Reinecke H, Gill E A, Nelson V, Kiem H P, Laflamme M A, Murry C E (2014) Human embryonic-stem-cell-derived cardiomyocytes regenerate non-human primate hearts. Nature 510(7504):273-277.

Liu Y W, Chen B, Yang X, Fugate J A, Kalucki F A, Futakuchi-Tsuchida A, Couture L, Vogel K W, Astley C A, Baldessari A, Ogle J, Don C W, Steinberg Z L, Seslar S O, Tuck S A, Tsuchida H, Naumova A, Lyu M S, Lee J, Hailey D W, Reinecke H, Pabon L, Fryer B H, MacLellan W R, Thies R S, Murry C E (2018) Human embryonic stem cell-derived cardiomyocytes restore function in infarcted hearts of non-human primates. Nature Biotech 36(7):597-605.

Romagnuolo R, Masoudpour H, Porta-Sanchez A, Qiang B, Barry J, Laskary A, Qi X, Masse S, Magtibay K, Kawajiri H, Wu J, Sadikov TV, Rothberg J, Panchalingham K M, Titus E, Ren-Ke L, Zandstra P W, Wright G A, Nanthakumar K, Ghugre N R, Keller G, Laflamme M A (2019) Human embryonic stem cell-derived cardiomyocytes regenerate the infarcted pig heart but induce ventricular tachyarrhythmias. Stem Cell Reports 12:1-15

Shiba Y, Gomibuchi T, Seto T, Wada Y, Ichimura H, Tanaka Y, Ogasawara T, Okada K, Shiba N, Sakamoto K, Ido D, Shiina T, Ohkura M, Nakai J, Uno N, Kazuki Y, Oshimura M, Minami I, Ikeda U (2016) Allogeneic transplantation of iPS cell-derived cardiomyocytes regenerates primate hearts. Nature 538(7625):388-391.

### Example 2: Anti-Arrhythmic Drugs Tested to for the Treatment of Engraftment Arrhythmias

Yucatan miniature pigs weighing 30-40 kg were used to model myocardial infarction. For this purpose, animals were subjected to a 90-minute occlusion of the left anterior descending coronary artery using a percutaneous transluminal coronary angioplasty balloon. An implanted electrocardiography device allowed continuous remote monitoring of the heart for the duration of the study. The hPSC-derived cardiomyocytes were administered two weeks after infarction, and the pigs were immunosuppressed to prevent immune rejection of the transplanted cells. In total, 500 million cardiomyocytes were delivered via percutaneous transendocardial injections. The control groups did not receive any anti-arrhythmic drugs while the treated groups were administered an anti-arrhythmic indicated in TABLE 1. A number of anti-arrhythmic therapies were tested to determine their efficacy in treating and preventing engraftment arrhythmias. TABLE 1 summarizes these results (below).

TABLE 1
ANTIARRHYTHMIC TRIALS

| Model tested | Anti-arrhythmic | Concentration | Result |
|---|---|---|---|
| Swine | Lidocaine (Ib) | 100 mg IV | Modest HR effect, rare |
| | | | cardioversion |
| Swine | Flecainide (Ic) | 2 mg/kg PO, | No response on HR or EA burden |
| | | 4 mg/kg PO, | |
| | | 6 mg/kg PO, | |
| | | 10 mg/kg PO | |
| Swine | Propafenone (Ic) | 1 mg/kg IV, | No response on HR, transient |
| | | 2 mg/kg IV, | cardioversion |
| | | 3 mg/kg IV | * Dose limited emesis |
| Swine | Amiodarone (III) | 150 mg IV | Modest HR effect, frequent |
| | | | cardioversion |
| Swine | Sotalol (III) | 1 mg/kg PO, | No response on HR or EA burden |
| | | 2 mg/kg PO, | |
| | | 4 mg/kg PO | |
| Swine | Metoprolol (β₁AR) | 5 mg IV, | Moderate HR effect (IV only), no |
| | | 25 mg PO BID, | response on EA burden |
| | | 50 mg PO BID, | |
| | | 75 mg PO BID | |
| Swine | Ivabradine (I_{f}) | 2.5 mg PO, | Robust dose-dependent HR |
| | | 5 mg PO BID, | effect* (PO only), no response on |
| | | 10 mg PO BID, | EA burden |
| | | 15 mg BID; | *Severe sinus bradycardia, R-on- |
| | | 1 mg/kg IV, | T event and polymorphic VT |
| | | 2 mg/kg IV | |
| Swine | Dantrolene (RyR₁) | 2 mg/kg IV | No response on HR or EA burden |
| Non-human | Verapamil (CCB) | | Sudden cardiac death |
| primate | | | |

When administered alone, amiodarone produced a moderate effect on reducing heart rate and provided cardioversion. Ivabradine was able to slow heart rate but had no major effect on reducing arrhythmia burden in the engraftment models. The additional anti-arrhythmics tested had moderate or no significant effects on heart rate or reducing arrhythmia burden in engraftment animals.

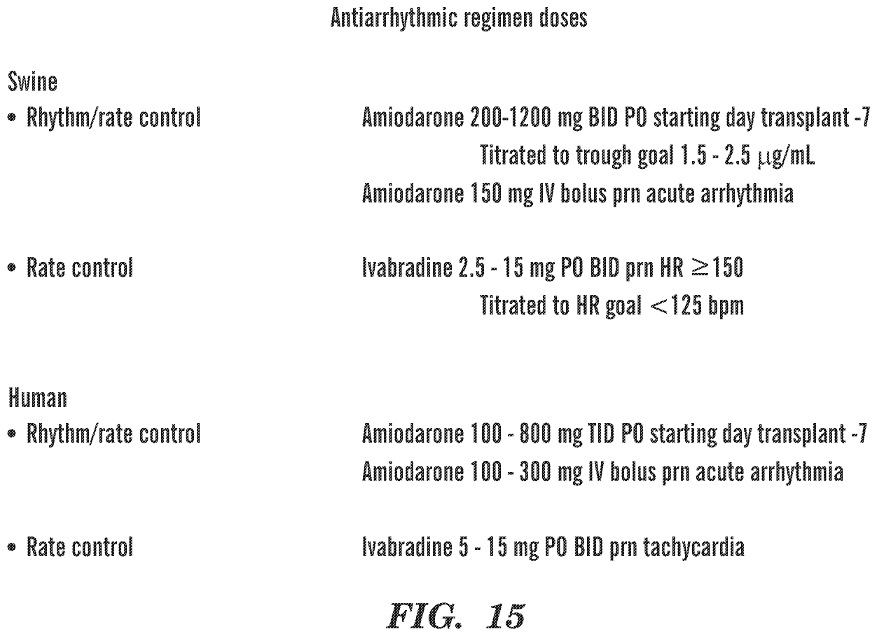

From the drugs tested, the combination of amiodarone and oral ivabradine was found to be the most effective in reducing both heart rate and arrhythmia burden in swine. In particular, the following regimens of amiodarone and ivabradine therapy were found to be the most efficacious in the engraftment animal model (see also FIG. 15).

### Swine

| | |
|---|---|
| Rhythm/rate | Amiodarone 200-1200 mg BID PO starting day |
| control | transplant −7 |
| | Titrated to trough goal 1.5-2.5 μg/mL |
| Rate control | Ivabradine 2.5-15 mg PO BID prn HR ≥150 |
| | Titrated to HR goal <125 bpm |

For comparison, the typical clinical dosages for amiodarone and ivabradine in human subjects are listed below:

### Human

| | |
|---|---|
| Rhythm/rate | Amiodarone 100-800 mg TID PO starting day transplant −7 |
| control | |
| Rate control | Ivabradine 5-15 mg PO BID prn tachycardia. |

In summary, the specific combination of amiodarone and ivabradine reduces heart rate and reduces engraftment arrhythmia burden in animals that received cell therapy compared with animals that were not treated with amiodarone and ivabradine (FIG. 1A-2).

### Example 3: Pharmacologic Therapy for Engraftment Arrhythmia Induced by Transplantation of Human Cardiomyocytes

Background Engraftment arrhythmias (EAs) are observed in large animal studies of intramyocardial transplantation of human pluripotent stem cell-derived cardiomyocytes (hPSC-CM) for myocardial infarction. Although transient, the risk posed by EA presents a barrier to clinical translation.

Methods hPSC-CM were transplanted into the infarcted porcine heart by surgical or percutaneous delivery to induce EA. Following a screen of antiarrhythmic agents, a prospective study was conducted to determine the effectiveness of amiodarone plus ivabradine in preventing cardiac death and suppressing EA.

Results EA was observed in all subjects, and amiodarone-ivabradine treatment was well tolerated. None of the treated subjects experienced the primary endpoint of cardiac death, unstable EA or heart failure compared to ⅝ (62.5%) in the control cohort (hazard ratio 0.00; 95% confidence interval, 0-0.297; p=0.002). Overall survival including two deaths in the treatment cohort from infection showed improvement with treatment (hazard ratio 0.21; 95% confidence interval, 0.03-1.01; p=0.05). Without treatment, peak heart rate averaged 305±29 beats/min, whereas in treated animals peak heart rate was significantly restricted to 185±9 beats/min (p=0.001). Similarly, treatment reduced peak EA burden from 96.8±2.9% to 76.5±7.9% (p=0.03). Antiarrhythmic treatment was safely discontinued after approximately one month of treatment without recrudescence of arrhythmia.

Conclusions The risk of engraftment arrhythmia and related sequelae following hPSC-CM transplantation can be reduced significantly by combined amiodarone and ivabradine drug therapy.

### INTRODUCTION

Myocardial infarction (MI) remains the leading cause of heart failure and death in the United States and around the world (1). During MI, approximately one billion cardiomyocytes are permanently lost, often leading to debilitating heart failure. Current treatments can slow the initiation and progression of heart failure, but none replaces lost myocardium, short of orthotopic heart transplantation, which remains restricted in availability and indication (2). Human pluripotent stem cells (hPSCs, comprising embryonic stem cells [ESCs] and their reprogrammed cousins, induced pluripotent stem cells (iPSCs) are a renewable source of cardiomyocytes (CMs). Transplantation of hPSC-derived cardiomyocytes (hPSC-CMs) into infarcted myocardium of small animals—mice, rats, guinea pigs—has shown stable engraftment (3-7). Remuscularization and functional benefit in infarcted non-human primates (NHP) following transplantation of human pluripotent stem cell hPSC-CM have been described (8-10). In addition to functional remuscularization, the human graft vascularizes and electromechanically couples with the host myocardium within one-month post-transplant and remains durable up to at least three months.

Although no arrhythmias were observed in smaller animals, the inventors and others consistently observe ectopic arrhythmias following hPSC-CM transplantation in NHPs (8-10) and pigs (11) which are termed engraftment arrhythmias (EAs) herein. EAs are generally transient, occurring within a week of transplantation and generally resolving spontaneously after one month. Based on electrical mapping, overdrive pacing, and cardioversion studies, EAs appear to originate focally in the graft or peri-graft myocardium and function as automatous foci rather than reentrant pathways (9,11). Although EA is reasonably well tolerated in NHPs, the Laflamme group (11) reported that EA can be lethal in some pigs. For this reason, EA has emerged as the biggest impediment to clinical translation of human cardiomyocyte transplantation (12).

Because the pig shows heightened sensitivity to EAs, and because it is a well-established model in cardiovascular research (13) and cell therapy (14), whose larger size permits use of percutaneous delivery catheters, approaches for mitigating the risk and sequellae of EA were tested in this large animal model. In the first phase of the study, a panel of anti-arrhythmic agents were screened. Amiodarone and ivabradine emerged independently as the most promising agents for control of rhythm and rate, respectively. A second phase to test was performed to determine the effect of combined amiodarone and ivabradine treatment. Interestingly, this regimen reduced sudden cardiac death, as well as suppressed tachycardia and arrhythmia.

### Methods

hESC-CM Production

Two lines of hESCs were used in this study. Initial subjects received H7 (WiCell)-derived cardiomyocytes that were cultured, expanded, and differentiated in suspension-culture as previously described (8,9,15). The majority of subjects received RUES2 (Rockefeller University)-derived cardiomyocytes in stirred suspension culture format. Briefly, RUES2 hESC were cultured to form aggregates and were expanded in commercially available media (Gibco Essential 8). For cardiac differentiation, suspension-adapted pluripotent aggregates were induced to differentiate in RPMI-1640 (Gibco), MCDB-131 (Gibco), or M199 (Gibco) supplemented with B-27 (Gibco) or serum albumin by timed use of small molecule GSK-3 inhibitors and Wnt/β-catenin signal pathway inhibitors (Tocris). Twenty-four hours prior to cryopreservation, RUES2 hESC-CMs were heat-shocked to enhance their survival after harvest, cryopreservation, thaw, and transplantation. Cardiomyocyte aggregates were dissociated by treatment with Liberase TH (Fisher) and TrypLE (Gibco) and were cryopreserved in CryoStor CS10 (Stem Cell Technologies) supplemented with 10 μM Y-27632 (Stem Cell Technologies) using a controlled-rate liquid nitrogen freezer. Approximately 3 h before transplantation, cryopreserved hESC-CM were removed from cryogenic storage (−150° C. to −196° C.) and thawed in a 37° C. water bath (2 min ±30 s). RPMI-1640 supplemented with B-27 and ≥200 Kunitz Units/mL DNase I (Millipore) was added to the cell suspension to dilute the cryopreservation media. Subsequent wash steps were done using RPMI-1640 basal media in progressively smaller volumes in order to concentrate the cell suspension. For the last centrifugation step, the cell pellet was resuspended in a sufficient volume of RPMI-1640 to achieve a target cell density for injection of ˜0.3×10⁹ cells/mL in 1.6 mL. In one case a larger dose of cells was resuspended in RPMI supplemented with serum albumin to a density of 0.43×10⁹ cells/mL in approximately 2.3 mL. The final volume of the cell suspension was determined by the results of a count sampled before the final centrifugation step. Cell counts were performed as described previously to achieve a final total dose of 500×10⁶ live cells (9).

### Study Design

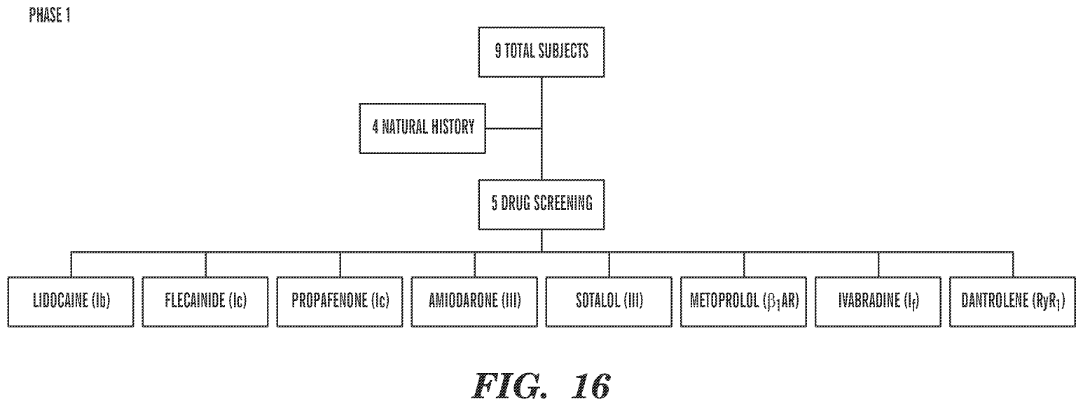

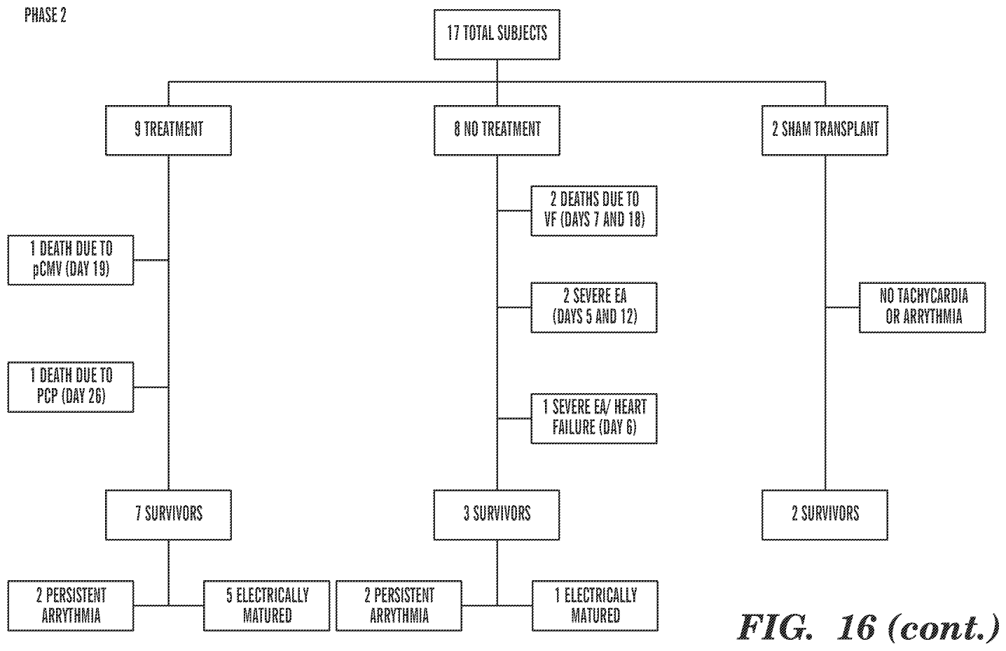

The objective of this study was to identify a pharmacological regimen to attenuate arrhythmias following cardiac remuscularization therapy. This study was designed in two phases: the first, to observe the natural history of EA and screen various antiarrhythmic agents for efficacy and the second, test for efficacy (FIG. 16). All subjects were 30-40 kg castrated male Yucatan minipigs between 6-13 months of age (S&S Farms). In Phase 1, nine subjects underwent cardiac remuscularization therapy with 500×10⁶ hESC-CM delivered by direct surgical transepicardial injections or later, by percutaneous transendocardial injections. The first four subjects (one non-infarcted and three infarcted) were followed to learn the natural history of EA and establish clinical endpoints and parameters for the Phase 2 drug trial. The subsequent five subjects underwent systematic dosing with antiarrhythmic agents with continuous electrocardiography (ECG) monitoring to determine effect on rhythm and rate (Table 2). Among the nine subjects in Phase 1, a high mortality was observed, with six out of nine experiencing ventricular fibrillation (VF) or tachycardia-induced heart failure requiring euthanasia. VF was associated with frequent non-sustained episodes of unstable EA >350 beats per minute (bpm) and heart failure with chronically elevated heart rate >150 bpm, which can be tachycardia-induced. Based on promising results from Phase 1, Phase 2 began as a prospective pilot drug trial to prevent EA-related mortality.

TABLE 2
Drug, dosage, and effects

| Drug | Dosage | Effect |
|---|---|---|
| Lidocaine (lb) | 100 mg IV | Modest HR effect, rare cardioversion |
| Flecainide (Ic) | 2 mg/kg PO, 4 mg/kg PO, 6 mg/kg PO, | No response on HR or EA burden |
| | 10 mg/kg PO | |
| Propafenone | 1 mg/kg IV, 2 mg/kg IV, 3 mg/kg IV | No response on HR, transient |
| (Ic) | | cardioversion* |
| Amiodarone | 150 mg IV | Modest HR effect, frequent |
| (III) | | cardioversion |
| Sotalol (III) | 1 mg/kg PO, 2 mg/kg PO, 4 mg/kg PO | No response on HR or EA burden |
| Metoprolol | 5 mg IV, 25 mg PO BID, 50 mg PO BID, | Moderate HR effect (IV only), no |
| (β₁AR) | 75 mg PO BID | response on EA burden |
| Ivabradine (I_{f}) | 2.5 mg PO, 5 mg PO BID, 10 mg PO BID, | Robust dose-dependent HR effect (PO |
| | 15 mg BID; 1 mg/kg IV, 2 mg/kg IV | only)**, no response on EA burden |

*Severe nausea/emesis observed at therapeutic doses, limiting clinical utility
**Severe bradycardia
Abbreviations:
β₁AK β1-adrenergic receptor;
BID, twice daily;
HR, heart rate;
EA, engraftment arrhythmia;
I_{f}, funny current;
PO, oral;
RyR₁, ryanodine receptor 1;
VT, ventricular tachycardia

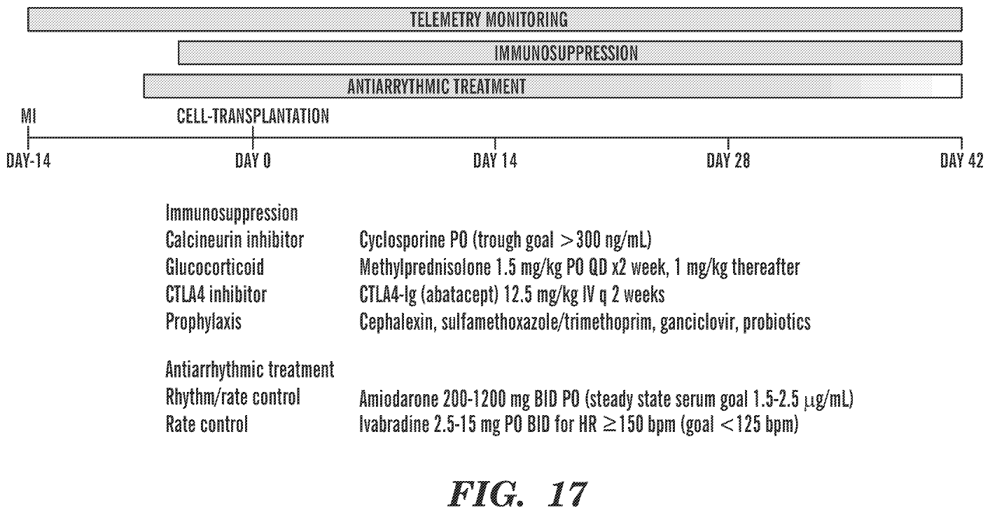

In Phase 2, a two-drug antiarrhythmic study was conducted with amiodarone and ivabradine and enrolled an additional 15 subjects (seven treatments, six untreated and two sham transplant) who underwent MI and percutaneous hESC-CM remuscularization at two weeks post-MI. Two additional subjects underwent MI with sham vehicle injection to serve as sham transplant controls (FIG. 16, FIG. 17). The primary endpoint was prespecified as combined cardiac death (either spontaneous death from arrhythmia or heart failure, or clinically directed euthanasia necessitated by tachycardia >350 bpm or signs of heart failure). Prespecified secondary endpoints were suppression of tachycardia, percent time in arrhythmia (arrhythmia burden) and resolution of arrhythmia, termed electrical maturation and defined as arrhythmia burden <25% for 48 consecutive hours. Antiarrhythmic therapy was discontinued after electrical maturation or post-transplantation day 30, whichever was earlier. To prevent excessive mortality, treatment was titrated to maintain target heart rate and arrhythmia burden at <150 bpm and <25%, respectively. Based on early experience that tachycardia >350 bpm often degenerated to VF, subjects were euthanized humanely if heart rates >350 bpm were reached. Continuous telemetric ECG was monitored for eight weeks total (two weeks post-MI and six weeks' post-transplantation). Of note, subjects 1 and 2 (no treatment) and 3 and 4 (treatment) received H7 hESC-CM and subjects 1, 2 and 3 were transplanted surgically prior to adopting percutaneous delivery. Subject 5 was euthanized on day 37 as a prespecified endpoint following electrical maturation, prior to extending the study duration to 6 weeks' post-transplantation for extended treatment washout and monitoring.

### Animal Care

All protocols were approved and conducted in accordance with the University of Washington (UW) Office of Animal Welfare and the Institutional Animal Care and Use Committee. Animals received ad libitum water and were fed twice a day (Lab Diet-5084 Laboratory Porcine Grower Diet). For surgical procedures, anesthesia was induced with a combination of intramuscular butorphanol, acepromazine and ketamine. Animals were intubated and mechanically ventilated using isoflurane and oxygen to maintain a surgical plane of anesthesia. Vital signs were monitored continuously throughout each procedure. All animals received subcutaneous Buprenorphine SR-Lab (ZooPharm) for post-operative analgesia and were euthanized by intravenous Euthasol (Virbac). All post-mortem examinations were performed by a blinded board-certified veterinary pathologist.

### Porcine Myocardial Infarction Model

Percutaneous ischemia/reperfusion injury was induced as previously described in NHP (9) with modification for the porcine model. A 5-8 cm incision was made in the femoral triangle and the femoral artery was exposed by blunt dissection. Prior to obtaining vascular access, heparin was administered to achieve therapeutic anticoagulation (activated coagulation time >250 sec). A 5-French guidewire/introducer sheath system (Terumo Medical) was placed into the femoral artery and secured. Continuous ECG, invasive arterial blood pressure, pulse-oximetry and capnography were monitored throughout the procedure. Intravenous amiodarone 150 mg and lidocaine 100 mg were administered as single boluses prior to ischemia to minimize the risk of arrhythmia. Under fluoroscopic guidance (OEC 9800 Plus, GE Medical Systems), a 5-French Judkins right 2 or hockey stick guide catheter (Boston Scientific) was advanced into the ascending aorta to selectively engage the ostium of the left main coronary artery. Coronary angiography was performed using hand injections of contrast (Visipaque) and a 0.035″ coronary guidewire (Runthrough NS Extra Floppy, Terumo Medical) was placed into the distal left anterior descending coronary artery (LAD). An appropriately sized angioplasty balloon catheter was then positioned into the mid-LAD distal to the first diagonal branch artery and inflated to the minimum pressure required for total obstruction of distal perfusion as confirmed by angiography. Ischemia was confirmed by ST-segment elevation on the ECG. Animals were maintained under anesthesia with ventilatory and hemodynamic support for 90 minutes, after which the balloon was deflated to restore distal perfusion, again confirmed by fluoroscopy and ECG. The animal was observed for reperfusion arrhythmias and externally cardioverted if ventricular fibrillation occurred. Prior to recovery, all subjects received implantable telemetry units and central venous catheter placement. Briefly, the external jugular vein in the jugular furrow was exposed and a 5-French central venous catheter (Access Technologies) was inserted and tunneled out to the dorsal prescapular area. The telemetry transmitter (EMKA easyTEL+) was implanted in a subcutaneous pocket using the same incision in the jugular furrow, and subcutaneous leads were tunneled to capture the cardiac apex to base. The overall procedural mortality including the infarct was <10%.

### Cardiac Remuscularization Therapy

Cell transplantation for three initial subjects (1-3) was performed by direct transepicardial injection into the peri-infarction region as previously described for NHP with minor modification (9). Briefly, a partial median sternotomy was performed to expose the infarcted anterior left ventricle. Purse-string sutures were preplaced at five discrete locations subtended by the LAD, targeting the central ischemic region and lateral border zones. After cinching the purse-string tightly around the needle, three injections of 100 μL each were performed by partial withdrawal and lateral repositioning, for a total of 15 injections to deliver total dose of 500×10⁶ hESC-CMs. All subsequent subjects (4-19) received cell transplantation via percutaneous transendocardial injection using the NOGA-MyoStar platform (BioSense Webster) to first map the infarct region in the left ventricle, and then to deliver 16 discrete endocardial injections of 100 μL each for total dose of 500×10⁶ hESC-CMs. Injections were only performed with excellent location and loop stability, ST-segment elevation and presence of premature ventricular contraction (PVC) with needle insertion in an appropriate location by electroanatomical map and unipolar voltage. For both surgical and percutaneous cell transplantation, two-thirds of injections were placed into the peri-infarct border zone defined visually or by unipolar voltage of 5-7.5 mV and the remaining one-third into the central ischemic region defined visuallly or as unipolar voltage of <5 mV. Two subjects (9 and 10) were infarcted as per protocol but received sham injections of RMI-1640 vehicle without cells to serve as sham transplant controls.

### Immunosuppression Therapy

All subjects received a three-drug immunosuppression regimen to prevent xenograft rejection as previously described with modification (9). For the initial regimen (subjects 1-6), five days prior to cell transplantation, oral cyclosporine A was started to maintain serum trough level of >400 ng/ml (approximately 250-1000 mg twice daily) for duration of the study. Two days prior to transplantation, oral methylprednisolone was started at 3 mg/kg for two weeks then titrated down to 1.5 mg/kg for the remainder of the study. On the day of transplantation, Abatacept (CTLA4-Ig, Bristol-Myers Squibb) 12.5 mg/kg was administered intravenously and dosed every two weeks thereafter. Due to complications related to immunosuppression (principally porcine cytomegalovirus and Pneumocystis pneumonia), the cyclosporine A trough level was decreased to >300 ng/ml and the methylprednisolone reduced to 1.0 mg/kg for subjects 7-19 without histologic evidence of rejection. Prophylactic oral cephalexin was administered for all subjects to prevent infection of the indwelling central venous catheter. Prophylactic sulfamethoxazole/trimethoprim was added after subject 3 developed Pneumocystis pneumonia. Prophylactic valganciclovir and probiotics were added after activation of endogenous porcine cytomegalovirus was found in subject 6.

### Antiarrhythmic Treatment

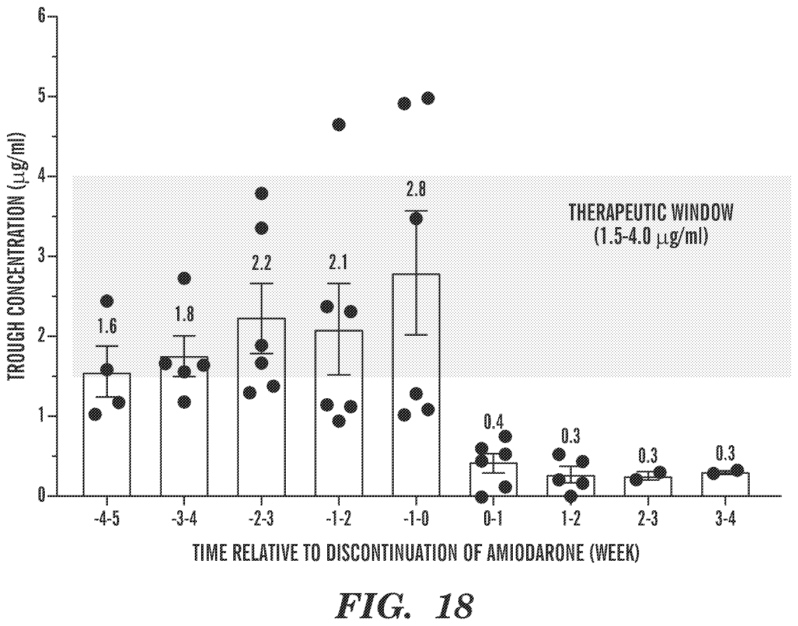

Treatment subjects were loaded with oral amiodarone 1000-1200 mg orally twice daily starting seven days prior to cell transplantation followed by maintenance dose of 400-1000 mg orally twice daily to maintain a steady-state plasma level of 1.5-4.0 μg/ml (FIG. 18). Ivabradine was started at 2.5 mg orally twice daily when sustained tachycardia reached ≥150 bpm and titrated every 3 days up to 15 mg twice daily for goal heart rate <125 bpm. All but one subject (subject 1) required adjunctive ivabradine for additional heart rate control. Antiarrhythmics were discontinued after electrical maturation was achieved or post-transplantation day 30, whichever was earlier, to allow for treatment washout and assess for recrudescence of arrhythmia. All subjects tolerated the antiarrhythmic regimen without complication. Untreated and sham transplant control subjects did not receive antiarrhythmic agents following the MI procedure, but otherwise received all immunosuppression and standard care.

### Amiodarone Drug Monitoring

A novel liquid chromatography—mass spectrometry assay was established for amiodarone to monitor steady state serum levels in the porcine model and guide oral dosing to ensure efficacy and avoid dose-related toxicity. A target serum level of 1.5-4.0 μg/ml was extrapolated from prior human pharmacokinetic studies (16,17). Elimination kinetics after discontinuation of oral amiodarone therapy were also studied by obtaining weekly trough concentrations in 6 pigs (subjects 6, 7, 8, 13, 14, 16) (FIG. 18).

### Electrocardiography (ECG) Analysis

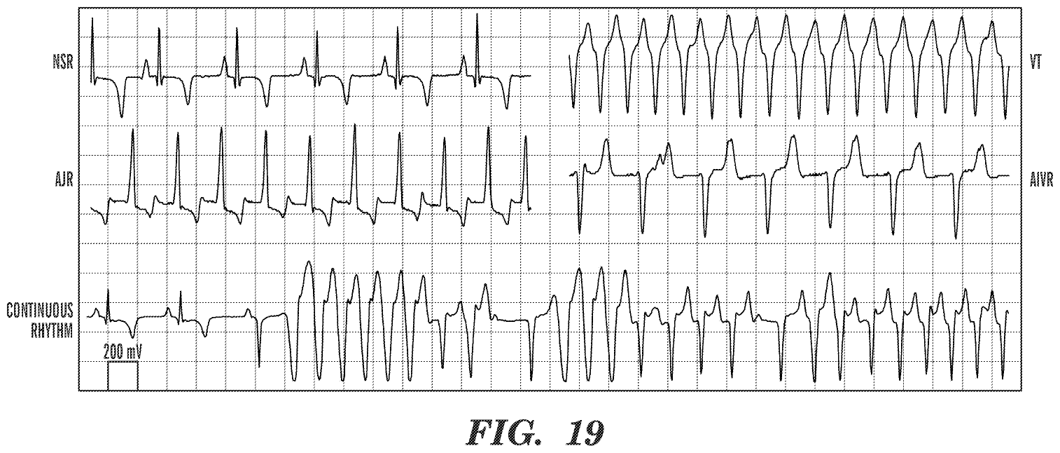

Telemetric ECG was continuously monitored in real-time from the time of myocardial infarction to detect the primary endpoint of cardiac death or unstable EA. Automated quantification of heart rate and arrhythmia burden was performed offline by a board-certified cardiologist using the ecgAUTO 3.3.5.10 software package (EMKA Technologies). Arrhythmia was defined as an ectopic beat (e.g. premature ventricular contraction) or rhythm (e.g. idioventricular rhythm, ventricular tachycardia). EA was typically observed as sustained and non-sustained ventricular tachyarrhythmia of varying rates and morphologies but also included slow and narrow complex ectopic rhythms (FIG. 19). Heart rate and arrhythmia burden were quantified for two continuous minutes every five minutes (40% of total rhythm was counted) and presented as daily averages.

### Histologic Analysis

Histological studies were carried out as detailed previously with modification (8,9). Briefly, paraformaldehyde-fixed hearts were dissected to remove the atria and right ventricle before short-axis cross-sections were cut at 2.5 mm intervals. The weights of the whole heart, left ventricle and each slice were obtained before further partition into tissue cassettes. The tissue then was processed, embedded in paraffin, and 4 μm sections were cut for staining. For morphometry, infarct regions were identified by picrosirius red staining; human graft was identified by anti-human cardiac troponin T, stained using avidin-biotin reaction (ABC Kit, VectorLabs) followed by chromogenic detection via diaminobenzidine (Sigmafast, Sigma Life Science) (FIG. 20). The slides were digitized using a whole slide scanner (Nanozoomer, Hamamatsu), and the images were viewed and exported with NDP.view 2.6.13 (Hamamatsu). Areas of infarct and graft were analyzed using a custom-written algorithm in the ImageJ open source software platform (18). Briefly, after extracting images in TIFF format (19), the image foreground was segmented by a threshold derived from the distribution in brightness of its pixels, resulting in a binary mask that delineates the imaged tissue section. Subsequent color de-convolution by thresholding hue, brightness and saturation allowed segmentation of regions stained by Picro-Sirius Red stain or areas immunolabelled for human cardiac troponin-T. To separate scar from diffuse fibrosis, a cut-off for particle size was applied. Infarct size and graft size were calculated as the (percent area x block weight), summed for the entire ventricle, and expressed as a percentage of left ventricular mass or infarct mass, respectively. Please see the Supplement Methods for Purkinje fiber staining

### Statistical Analysis

Statistical analyses and graphing were performed using Prism 8.4.2 software (GraphPad) and Stata 15 (StataCorp, College Station, Tex.). Data are presented as mean±standard error of the mean (SEM). Comparisons were performed using two-tailed Student t-test with significance threshold of P<0.05 unless otherwise specified. Error bar plots show how the mean±SEM of heart rate and arrhythmia burden varies over time in the two treatment groups. Kaplan-Meier plots show survival curves for the primary endpoint of cardiac death, unstable EA or heart failure, and for all-cause mortality. Cox proportional regression models are used to estimate the hazard ratio (HR) between the two treatment groups, for the primary outcome and for mortality. Significance is based on the likelihood ratio test and confidence intervals on HR are computed by inverting the likelihood test, based on varying the offset term in the stcox procedure in Stata.

### Results

Percutaneous Delivery of hESC-CM in Infarcted Porcine Model

Catheter-based endocardial delivery of hESC-CM was safe and effective in remuscularizing the infarcted porcine heart (FIG. 20). No significant differences in myocardial infarct or cardiomyocyte graft sizes were observed between the treatment groups. The average infarct size for the treatment and no-treatment cohorts were comparable at 11.7±1.1% and 10.5±2.0% of the left ventricle, respectively (p=0.59). Graft size relative to infarct size was also comparable at 2.3±0.7% and 2.8±1.3% for treatment and no treatment, respectively (p=0.74). Delivery of hESC-CM successfully targeted the peri-infarct border zone and central ischemic regions as intended and resulted in discrete hPSC-CM grafts transplanted into host myocardium as previously reported (8-11). All grafts were located in the anterior, antero-septal and antero-lateral walls and appeared structurally immature at early time points before 2 weeks' post-transplantation with increasing maturity up to the end of study as previously reported in pig (11).

### Clinical History of Engraftment Arrhythmia

A flow chart for all animals in the study is shown in FIG. 16. No significant arrhythmias were noted in the two sham transplant subjects (9 and 10) that underwent myocardial infarction and percutaneous intracardiac injection of vehicle. All subjects that received human cardiomyocyte grafts developed EA between 2-6 days following cell transplantation. Initiation of EA was characterized by salvos of non-sustained VT, and this typically progressed to periods of sustained VT with rates ranging from 110 to 250 bpm (FIG. 19). The VT was often polymorphic, with the same animal showing different electrical axes and both wide- and narrow-complex tachycardia at different times. In 4 of the 8 untreated animals, EA was either fatal or necessitated euthanasia due to a prespecified endpoint of unstable tachycardia (defined as sustained heart rate >350 bpm). In one additional untreated case (subject 12), acute heart failure was noted clinically shortly after initiation of EA at a rate of 300 bpm and based on recommendations from veterinary staff, the subject was euthanized. Signs of heart failure were subsequently confirmed on necropsy. In all other cases, EA was noted with a rapid acceleration to >350 bpm (subjects 11 and 12) and, in two cases, deterioration to VF prior to euthanasia (subjects 1 and 2) (Table 3). Three out of four arrhythmic endpoints occurred within the first three days of developing EA, and they occurred when tachyarrhythmia was nearly constant. Mean heart rate peaked at day 8 post-transplantation and began to decline after that, whereas the arrhythmia burden plateaued from days 8-16 and began to normalize thereafter. Of the three survivors in the untreated cohort, two did not normalize rhythm and experienced on average 42% arrythmia burden at the end of study (subjects 15 and 17). The single subject in the untreated cohort that normalized heart rate and rhythm did so on post-transplant day 26 (subject 11).

TABLE 3
Subject Outcomes

| | | | | | | | | | Graft | | | Arrhythmia | | |
|---|---|---|---|---|---|---|---|---|---|---|---|---|---|---|
| | | | | | | | Via- | Infarct | size - | | | burden - % | | |
| Sub- | Age - | Weight - | | Cell | Ap- | cTnT - | bility - | size - | % | HR - bpm | | of day | | |
| ject | mo | kg | MI | line | proach | % | % | % LV | Infarct | Day 7 | Day 30 | Day 7 | Day 30 | Outcome |

Treatment

| | | | | | | | | | | | | | | |
|---|---|---|---|---|---|---|---|---|---|---|---|---|---|---|
| 3 | 7.7 | 34.7 | Yes | H7 | Surg | 98% | 89% | — | — | 72.8 | n/a | 2.0 | n/a | Euthanasia, |
| | | | | | | | | | | | | | | day 26 (PCP) |
| 4 | 7.6 | 32.0 | Yes | H7 | Perc | 98% | 89% | — | — | 92.7 | 93.9 | 86.0 | 97.8 | Survival |
| 5 | 7.9 | 33.0 | Yes | RUES2 | Perc | 91% | 89% | — | — | 162.6 | 80.9 | 25.6 | 0.5 | Survival |
| 6 | 7.7 | 33.4 | Yes | RUES2 | Perc | 91% | 90% | 10.1% | 1.4% | 99.6 | n/a | 47.1 | n/a | Euthanasia, |
| | | | | | | | | | | | | | | day 19 (pCMV) |
| 7 | 7.7 | 37.0 | Yes | RUES2 | Perc | 91% | 93% | 9.6% | 0.3% | 89.4 | 78.6 | 45.8 | 28.2 | Survival |
| 8 | 10.0 | 33.5 | Yes | RUES2 | Perc | 86% | 88% | 15.9% | 3.4% | 72.8 | 77.4 | 67.6 | 4.7 | Survival |
| 15 | 9.0 | 33.0 | Yes | RUES2 | Perc | 89% | 88% | 10.9% | 4.2% | 79.4 | 75.7 | 35.4 | 44.9 | Survival |
| 16 | 8.8 | 34.0 | Yes | RUES2 | Perc | 88% | 85% | 14.6% | 0.7% | 74.2 | 68.6 | 43.9 | 7.6 | Survival |
| 18 | 12.7 | 32.5 | Yes | RUES2 | Perc | 94% | 75% | 9.3% | 3.7% | 69.0 | 78.2 | 1.6 | 1.6 | Survival |
| Avg ± | 8.8 ± | 33.7 ± | | | | 90 ± | 87 ± | 11.7 ± | 2.3 ± | 90.3 ± | 79 ± | 39.5 ± | 26.5 ± | |
| SEM | 0.6 | 0.5 | | | | 1% | 2% | 1.1% | 0.7% | 9.7 | 2.9 | 9.2 | 13.4 | |

No Treatment

| | | | | | | | | | | | | | | |
|---|---|---|---|---|---|---|---|---|---|---|---|---|---|---|
| 1 | 8.9 | 32.0 | No | H7 | Surg | 98% | 88% | — | — | 328.2 | n/a | 100.0 | n/a | Primary |
| | | | | | | | | | | | | | | endpoint |
| | | | | | | | | | | | | | | day 7 (VF) |
| 2 | 10.5 | 32.0 | Yes | H7 | Surg | 98% | 90% | — | — | 120.2 | n/a | 62.7 | n/a | Primary |
| | | | | | | | | | | | | | | endpoint |
| | | | | | | | | | | | | | | day 18 (VF) |
| 11 | 9.4 | 33.5 | Yes | RUES2 | Perc | 82% | 87% | 9.7% | 1.1% | 165.6 | n/a | 94.0 | n/a | Primary |
| | | | | | | | | | | | | | | endpoint |
| | | | | | | | | | | | | | | day 12 (EA) |
| 12 | 9.5 | 35.0 | Yes | RUES2 | Perc | 86% | 90% | 13.2% | 1.0% | n/a | n/a | n/a | n/a | Primary |
| | | | | | | | | | | | | | | endpoint |
| | | | | | | | | | | | | | | day 5 (EA) |
| 13 | 9.7 | 35.5 | Yes | RUES2 | Perc | 87% | 83% | 16.5% | 1.7% | 162.0 | 82.6 | 95.9 | 1.9 | Survival |
| 14 | 9.0 | 33.0 | Yes | RUES2 | Perc | 87% | 90% | 4.6% | 9.1% | n/a | n/a | n/a | n/a | Primary |
| | | | | | | | | | | | | | | endpoint |
| | | | | | | | | | | | | | | day 6 (EA/HF) |
| 17 | 7.0 | 33.0 | Yes | RUES2 | Perc | 98% | 70% | 13.5% | 0.4% | 106.0 | 115.2 | 49.1 | 75.7 | Survival |
| 19 | 6.4 | 33.5 | Yes | RUES2 | Perc | 92% | 74% | 5.4% | 3.6% | 98.5 | 84.3 | 71.5 | 51.7 | Survival |
| Avg ± | 8.8 ± | 33.4 ± | | | | 91 ± | 84 ± | 10.5 ± | 2.8 ± | 163.4 ± | 94 ± | 78.9 ± | 43.1 ± | |
| SEM | 0.5 | 0.4 | | | | 2% | 3% | 2% | 1.3% | 34.9 | 10.6 | 8.5 | 21.7 | |
| P- | 0.97 | 0.73 | | | | 0.77 | 0.31 | 0.59 | 0.74 | 0.03 | 0.09 | 0.01 | 0.52 | |
| value^{†} | | | | | | | | | | | | | | |

Sham Transplant

| | | | | | | | | | | | | | | |
|---|---|---|---|---|---|---|---|---|---|---|---|---|---|---|
| 9 | 8.30 | 33.5 | Yes | RUES2 | Perc | n/a | n/a | — | n/a | 71.5 | 67.6 | 0.8 | 0.0 | Survival |
| 10 | 7.77 | 33.0 | Yes | RUES2 | Perc | n/a | n/a | — | n/a | 76.5 | 69.4 | 1.2 | 0.8 | Survival |
| Avg ± | 8.0 ± | 33.3 ± | | | | | | | | 74 ± | 68.5 ± | 1.0 ± | 0.4 ± | |
| SEM | 0.3 | 0.3 | | | | | | | | 2.5 | 0.9 | 0.2 | 0.4 | |

^{†}Treatment vs No treatment
Abbreviations:
bpm, beats per minute;
EA, engraftment arrhythmia;
hESC-CM, human embryonic stem cell-derived cardiomyocytes;
HF, heart failure;
HR, heart rate;
MI, myocardial infarction;
surg, surgery;
PCP, pneumocystis pneumonia;
pCMV, porcine cytomegalovirus;
perc, percutaneous;
VF, ventricular fibrillation

### Screening Drugs for Anti-Arrhythmic Effects

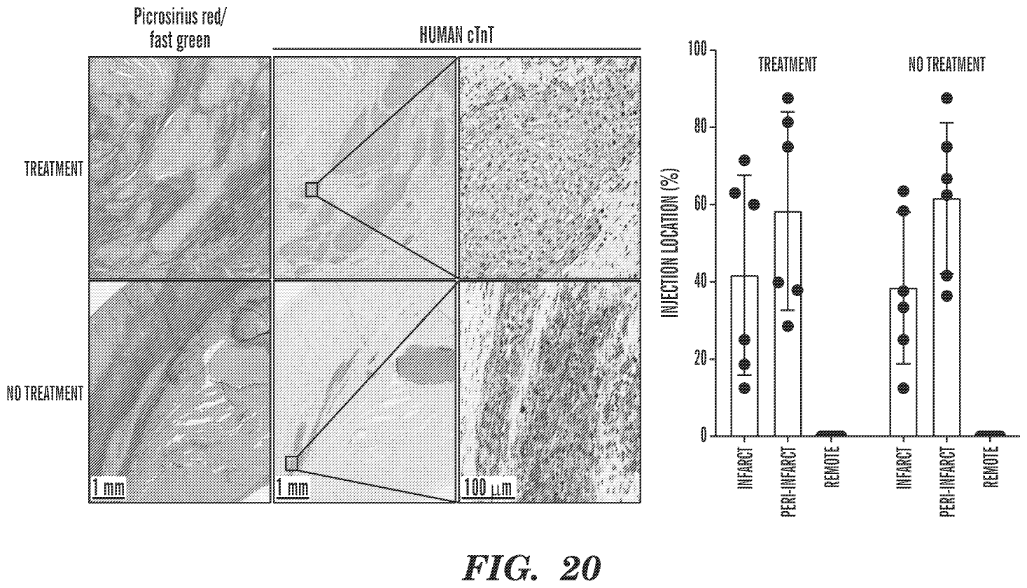

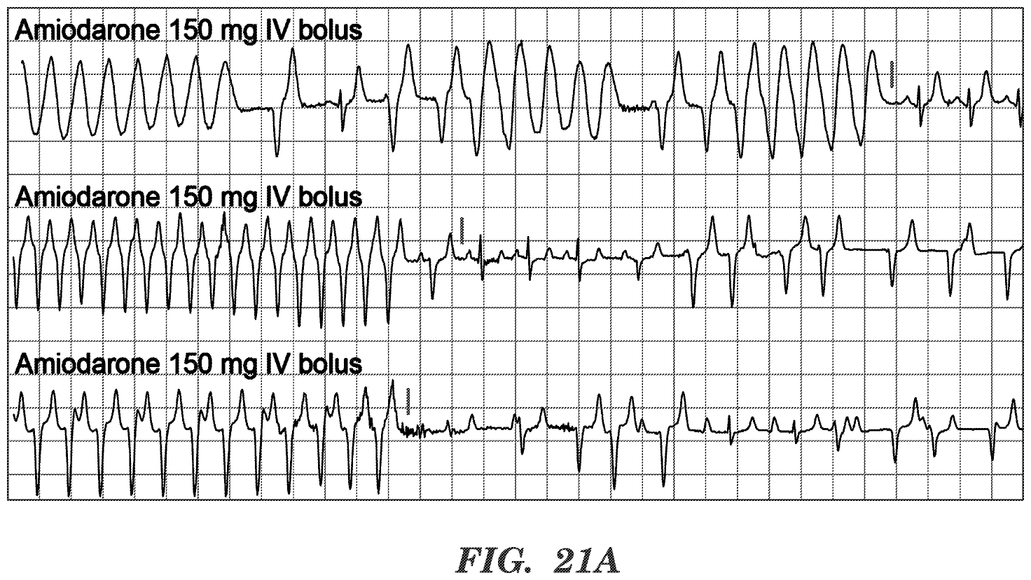

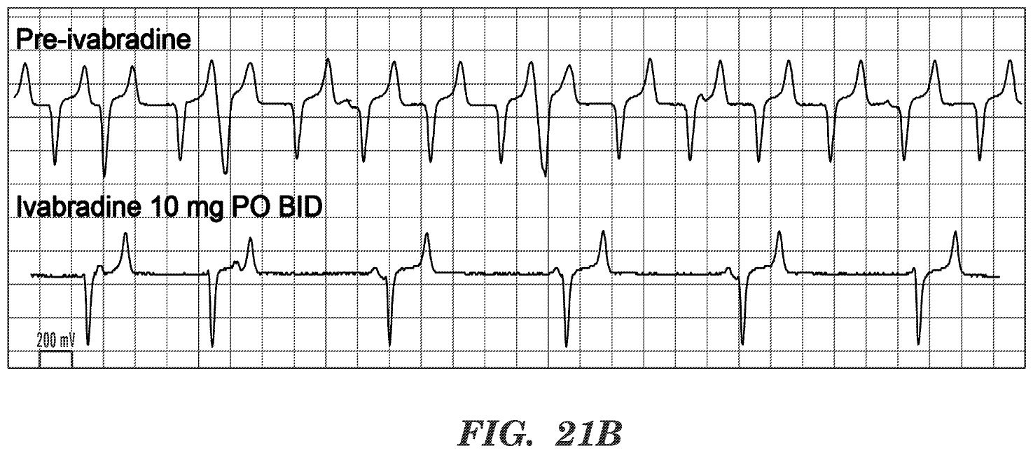

In Phase 1 of the study, six canonical antiarrhythmic agents broadly targeting sodium channels, potassium channels, and beta-adrenergic receptors were screened: lidocaine (Ib, sodium channel inhibitor), flecainide (Ic, sodium channel inhibitor), propafenone (Ic, sodium channel inhibitor), amiodarone (III, potassium channel inhibitor), sotalol (III, potassium channel inhibitor) and metoprolol β1-adrenergic receptor inhibitor) for effect on EA heart rate and rhythm. In addition, the funny current/HCN4 channel antagonist, ivabradine, was tested (Tables 1 and 2). This series was not meant to be definitive but rather to rapidly identify candidate agents. Animals were brought into the laboratory while in EA, anesthetized, and the effects of short-term intravenous infusion of anti-arrhythmic agents were studied. In three instances, intravenous amiodarone successfully cardioverted unstable EA from >350 bpm to a lower heart rate typically to sinus rhythm, albeit briefly (FIG. 21A). Oral ivabradine demonstrated robust dose-dependent effects on heart rate, but it did not restore sinus rhythm (FIG. 21B). Five of the other drugs had no significant effect in this screen (lidocaine, flecainide, sotalol, and metoprolol). Propafenone briefly reduced heart rate and restored sinus rhythm in two drug challenges, but this drug was associated with substantial gastrointestinal toxicity and not studied further (data not shown).

### Amiodarone-Ivabradine Enhance Survival

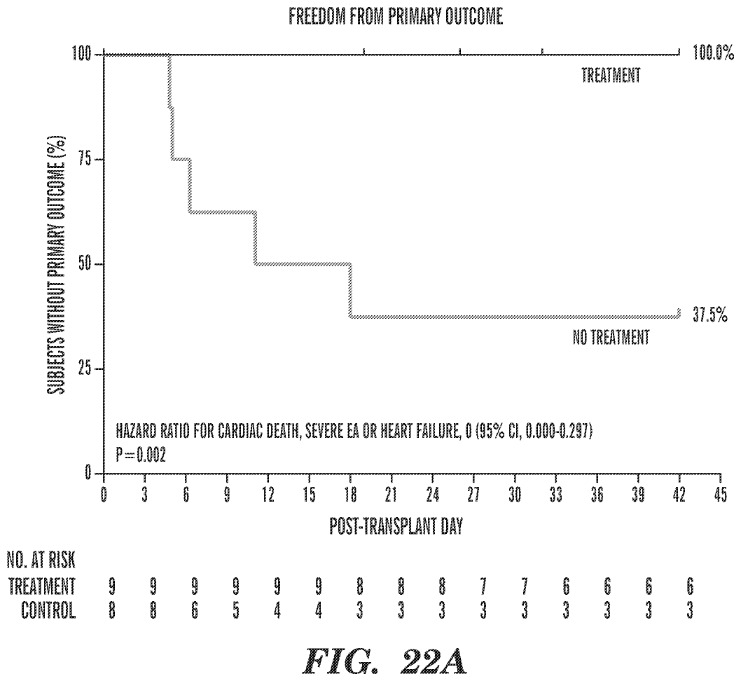

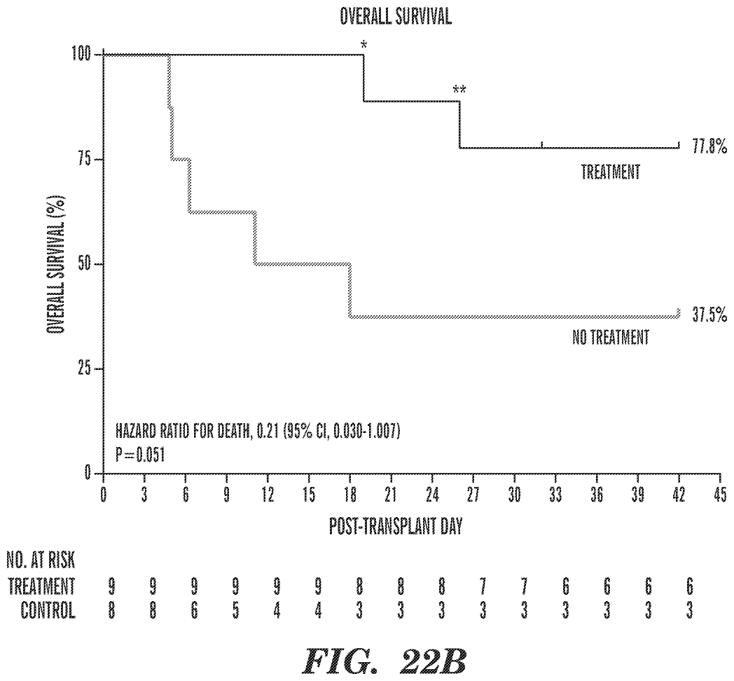

It was contemplated that chronic amiodarone with adjunctive ivabradine would reduce a combined primary endpoint of cardiac death, unstable EA >350 bpm and heart failure in Phase 2 of the study. A total of nine treated, eight untreated, and two sham transplant subjects were enrolled in the study with similar baseline and cell transplantation characteristics (Table 3). As detailed in Methods, treated animals received bolus and maintenance doses of amiodarone, and ivabradine was given as needed to keep heart rates <150 bpm. All treatment subjects (100%) survived without the primary cardiac endpoint compared to ⅜ (37.5%) of untreated subjects (FIG. 22A). The hazard ratio of the primary endpoint was 0.000 (95% CI, 0.000-0.297; p=0.002) with antiarrhythmic treatment. Of note, two of the treatment subjects (3 and 6) experienced non-cardiac deaths at post-transplant days 19 and 26 due to immunosuppression-related complications (Pneumocystis pneumonia and porcine cytomegalovirus, respectively). Intention-to-treat analysis of overall survival also favored the treatment cohort with hazard ratio of 0.212 (95% CI, 0.030-1.007; p=0.051) (FIG. 22B).

### Suppression of Tachycardia and Arrhythmia Burden

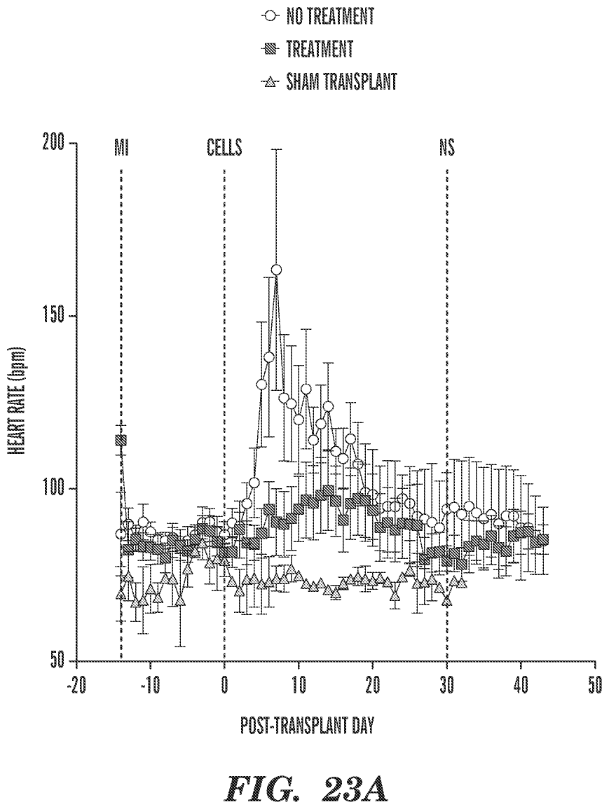

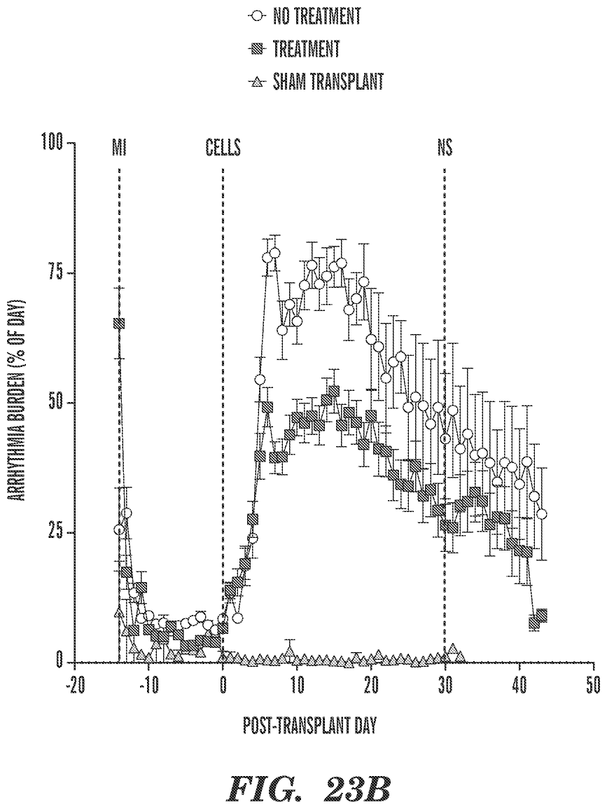

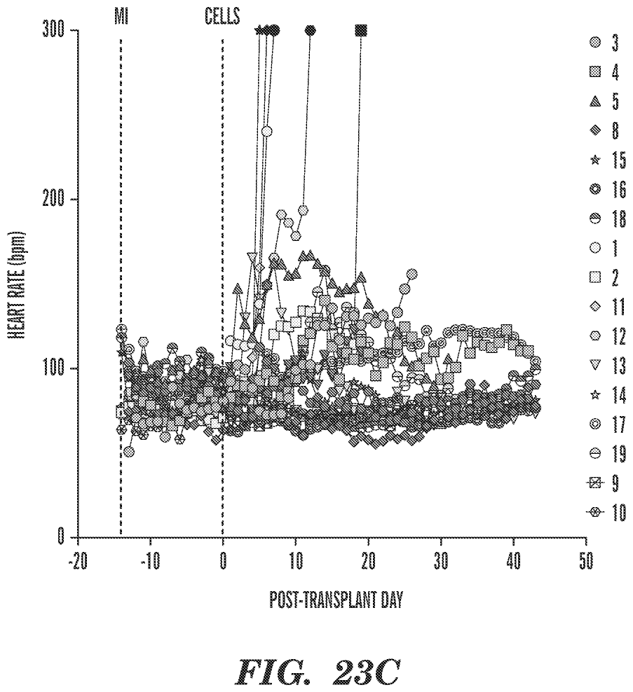

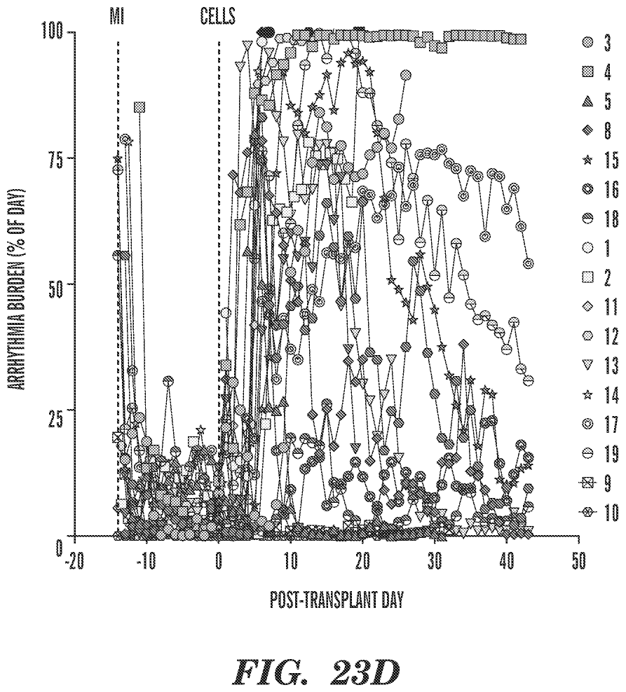

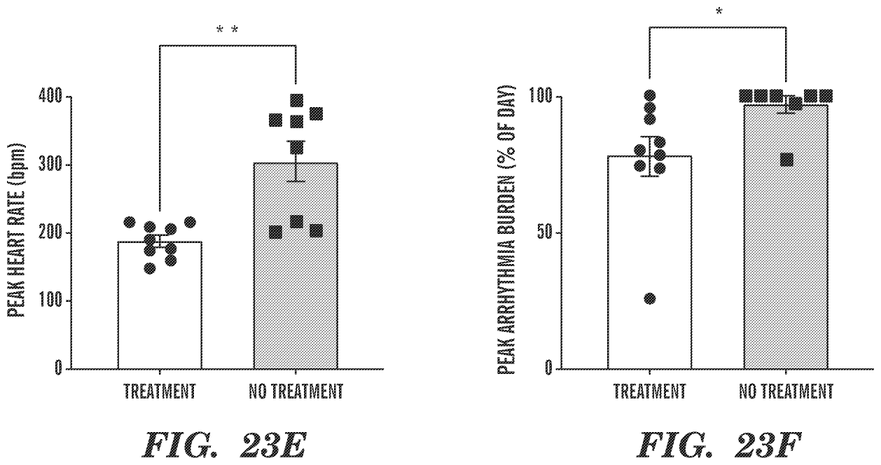

Pooled and individual subject-level data of heart rate and arrythmia burden are provided in FIGS. 23A-23B and 23C-23D, respectively. The average heart rate was significantly lower with antiarrhythmic treatment compared to no treatment. Mean heart rates peaked at post-transplantation day 7 in untreated animals at 163±35 bpm, when in the treated group heart rates averaged 90±10 bpm (p=0.03) (Table 3 and FIG. 23). Heart rate in the treated animals was not significantly different than the normal resting heart rate prior to MI and transplant (84±1 bpm, p=0.21). Following transplantation, peak heart rate averaged 305±29 beats/min in untreated animals, whereas treatment significantly restricted tachycardia to 185±9 beats/min (p=0.001) (FIG. 23E). Arrhythmia burden was defined as the percentage of the day spent in arrhythmia. Treatment reduced peak arrhythmia burden from 96.8±2.9% to 76.5±7.9% (p=0.03) (FIG. 23F). No differences in heart rate or arrhythmia burden were noted at post-transplant day 30, as the majority of arrhythmia had resolved irrespective of treatment (FIG. 23A-23B) (p=0.09 and p=0.52, respectively).

Antiarrhythmic treatment was safely discontinued by day 30 in all treatment subjects who achieved electrical maturation without recrudescence of arrhythmia (FIG. 23). Two treated and two untreated subjects (3, 4 and 15, 17, respectively) failed to mature electrically and exhibited significant arrhythmia at the end of study. In these four animals, heart rates were well controlled irrespective of treatment, and they survived until the study's completion. Average serum amiodarone was sub-therapeutic at 0.42±0.12 μg/ml within 1 week of discontinuation (FIG. 19).

Graft Interaction with Host Purkinje Conduction System

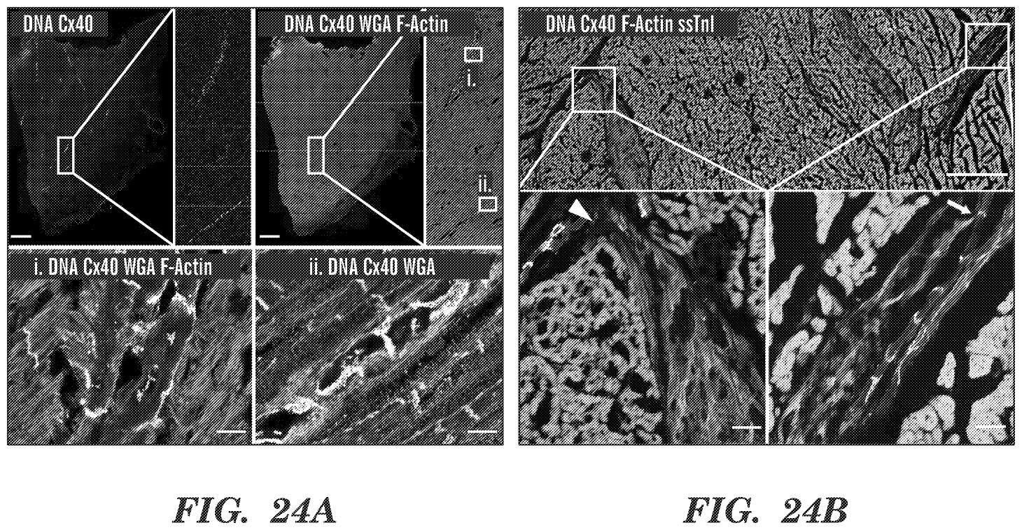

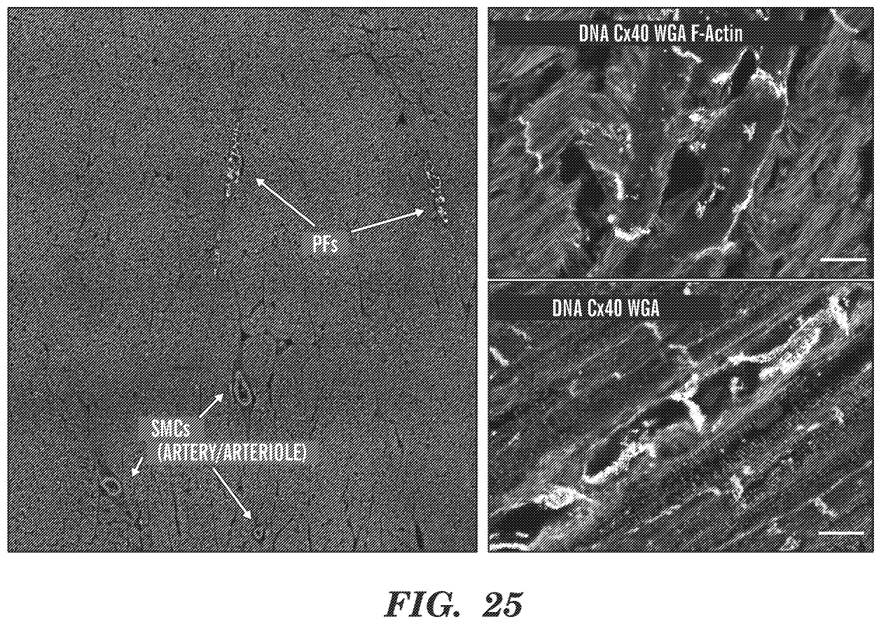

Microscopy of hESC-CM graft in porcine myocardium demonstrates interaction with the diffuse Purkinje conduction system of the host porcine heart (FIG. 24). Consistent with previous reports (20,21), a mesh-like network of intramural Purkinje fibers (PFs) throughout the left ventricle (FIG. 24A; video data not shown confirmed the mesh-like network throughout the native porcine myocardium) which localize with hESC-CM grafts in close proximity were observed (FIG. 24B; video data not shown confirmed that hPSC-cardiomyocytes, marked by slow skeletal troponin I (ssTn1), interact with connexin 40+purkinje fibers). Connexin 40 (Cx40) specifically stains Purkinje cell gap junctions (20,22) where lower sarcomere content and lack of T-Tubules are observed as expected (FIG. 25).

### DISCUSSION

Intramyocardial transplantation of hPSC-CM is a promising strategy to remuscularize the infarcted heart and restore function (2). Such a therapy to prevent and treat heart failure would be a seminal advance in addressing a large unmet need. Studies in large animals have demonstrated long-term efficacy but also defined a significant safety signal of transient but potentially fatal arrhythmias. As demonstrated in earlier studies (9-11), EA is a predictable complication of cardiac remuscularization therapy for myocardial infarction (23). In the NHP, EA typically presents as a wide-complex tachycardia with a variable electrical axis (8,9), and this was reproduced in the pig recently by the Laflamme laboratory (11). EA is described herein as polymorphic due to the observed changes in electrical axis as ectopy originating from different graft foci. Interestingly, in the pig a narrow-complex VT was observed that alternates with wide-complex tachycardia, a pattern not seen in the NHP. Without wishing to be bound by theory, histology of native and grafted porcine myocardium support the hypothesis that that the wide-complex beats originate from grafts contacting the working-type myocardium with slow conduction, and that the narrow complex beats originate when grafts contact the intramural Purkinje fibers that diffusely permeate the porcine heart (20,21). All 17 subjects transplanted with 500×10⁶ hESC-CMs demonstrated significant burden of arrhythmia that, while typically transient, was associated with high mortality in pigs. A higher morbidity and mortality related to EA was observed in this study when compared with the recent study by Laflamme and colleagues (11). This reflects differences in the Yucatan minipig model, percutaneous cell delivery, or the cell product. The experiments described herein suggest two primary mechanisms of cardiac morbidity. First, rapid EA >350 bpm can degenerate to fatal ventricular fibrillation, and second, heart failure commonly ensued in pigs with chronic tachycardia >230 bpm (24). Consequently, the primary endpoint included these parameters to limit excessive mortality in the antiarrhythmic trial.

Combined antiarrhythmic treatment with baseline amiodarone and adjunctive ivabradine safely prevented the combined primary endpoint of cardiac death, unstable EA and heart failure in all treated subjects, indicating that the risk of EA can be mitigated through pharmacology. Treatment was associated with significantly decreased peak tachycardia and arrhythmia. Once subjects experienced sustained improvement in arrhythmia burden, termed electrical maturation, antiarrhythmic therapy was successfully withdrawn in all subjects. Thus, short-term amiodarone and ivabradine treatment promoted electrical stability until the grafts became less arrhythmogenic.

While not wishing to be bound by theory, the mechanism of benefit for the antiarrhythmic treatment may be related to suppression of automaticity, reducing both heart rate and arrhythmia burden. The drugs were particularly beneficial during the early phase of EA, which carries the greatest risk of deterioration to VF. Electrophysiological studies performed in NHP (9) and pig (11), respectively, suggests that the etiology of EA is increased focal automaticity, rather than macro-reentry typically observed with clinical ventricular tachycardia (25). As EA became unstable in the untreated animals, heart rates rapidly accelerate to >350 bpm, and the possibility that this escalation can be a distinct mechanism, e.g. automaticity leading to reentry, was not excluded. This suggests why treatment successfully suppressed unstable and fatal arrhythmias but was unable to prevent EA altogether. The efficacy of ivabradine to rate-control EA suggests that its pharmacologic target, the I_{f} current carried by the HCN4 channel, which is highly expressed in immature cardiomyocytes and hPSC-CMs (26), can be an important mediator. Ivabradine, by itself, never abrogated EA, suggesting that the I_{f} current is a rate-modulator but not the sole source of the arrhythmia. In contrast, amiodarone reduced the burden of EA chronically and clearly restored sinus rhythm in some acute infusion experiments (FIG. 21A-21B). Although classified principally as a K⁺ channel blocker, amiodarone is well-known also to antagonize Na⁺channels, Ca²⁺channels, and β-adrenergic receptors (27). Thus, while in no way diminishing the importance the discovery of the effective drug combination disclosed herein, it is difficult to gain insights into the mechanism of EA from amiodarone's efficacy. The disappearance of EA coincides with maturation of the stem cell-derived graft (8,9,28) and it is contemplated that the window of arrhythmogenicity can reflect a period of in vivo graft maturation prior to reaching a state more similar to host myocardium (26,29-33). Additional strategies such as promoting maturation prior to transplanting, gene editing, and modulating host/cell interaction can provide additional means of arrhythmia control. Further investigation of the mechanism underlying EA would be accelerated by the development of higher throughput platforms to perform genetic, pharmacological and electrophysiological studies before phenotyping in large animal models.

Engraftment arrhythmia is the most significant barrier to clinical translation of cardiac remuscularization therapy. The natural history of EA emerging from the NHP and more recent porcine data suggests that, once EA resolves, there is low risk for further arrhythmia. This study provides a proof-of-concept that clinically relevant antiarrhythmic drug treatment can successfully suppress fatal arrhythmias and control tachycardia to achieve electrical quiescence. This is an important step forward in regard to the clinical safety of cardiomyocyte transplantation for cardiac remuscularization therapy.

This study demonstrates that EA is responsive to pharmacologic suppression. Clinically relevant doses of amiodarone and ivabradine were administered in the study.

### CONCLUSIONS

In this study utilizing a porcine infarction model of cardiac remuscularization therapy, EA was universally observed and associated with significant mortality. Chronic amiodarone treatment combined with adjunctive ivabradine successfully prevented the combined primary endpoint of cardiac death, unstable EA and heart failure. Overall survival was significantly improved with antiarrhythmic treatment and associated with heart rate and rhythm control.

### Example 5: Supplemental Methods

### Pilot Antiarrhythmic Screening in Pig

Five infarcted pigs underwent hESC-CM transplantation with all exhibiting stereotypic EA. Subjects were administered multiple trials antiarrhythmics and observed for acute response by continuous ECG monitoring. Intravenous agents were delivered as a bolus dose over 2 minutes. Oral agents were administrated by direct observation in a minimum of apples, apple sauce or pumpkin puree with daily feeding and dosed daily for dose escalation. A washout period of at least three days was provided between agents. Amiodarone was administered as the last agent for testing given concern for prolonged half-life and elimination kinetics. All agents were tested in at least two subjects.

### Purkinje Fiber Histology

For thin sections, tissue was cut and trimmed to 1 cm×1 cm×3 mm, snap frozen in isopentane, and embedded in OCT (TissueTek). 10 μm sections were immersed in 100% methanol at −20° C. for 15 minutes and stained with standard immunofluorescence technique using stains described below. Images were acquired on a Leica SP8 confocal microscope.

For thick sections, 1 cm×1 cm×3 mm pieces of tissue containing graft were incubated in 100% methanol at 20° C. for 1 hour, rehydrated (80% methanol, 60% methanol, 0% methanol, diluted in PBS, 15-minute incubation at −20° C. for each reagent). 150 μm sections were cut on a Leica VT1200s vibratome and stained with standard immunofluorescence technique using stains described below. Stained sections were then cleared using BABB as previously reported (34), and imaged on a Leica SP8 confocal microscope with 1 μm z-step increments.

### Purkinje Fiber Staining

Sections were stained the following reagents: Hoechst 33342 (DNA, Thermo Fisher Scientific, #62249), Wheat germ agglutinin-Oregon Green (WGA, Thermo Fisher Scientific, #W6748), Phalloidin-647 (F-Actin, Thermo Fisher Scientific, #A22287), anti-Connexin 40 (Cx40, Alpha Diagnostics, #CXN40A), or anti-slow skeletal troponin I (ss-TnI, Novus, #NBP2-46170) with one of two anti-rabbit secondary antibodies (Alexa Fluor 555/647, Thermo Fisher Scientific, #A-31570/A-31573).

### REFERENCES

1. Collaborators GBDCoD. Global, regional, and national age-sex-specific mortality for 282 causes of death in 195 countries and territories, 1980-2017: a systematic analysis for the Global Burden of Disease Study 2017. Lancet 2018; 392:1736-1788.
2. Nakamura K, Murry C E. Function Follows Form-A Review of Cardiac Cell Therapy. Circ J 2019; 83:2399-2412.
3. Caspi 0, Huber I, Kehat I et al. Transplantation of human embryonic stem cell-derived cardiomyocytes improves myocardial performance in infarcted rat hearts. J Am Coll Cardiol 2007; 50:1884-93.
4. Laflamme M A, Chen K Y, Naumova A V et al. Cardiomyocytes derived from human embryonic stem cells in pro-survival factors enhance function of infarcted rat hearts. Nat Biotechnol 2007; 25:1015-24.
5. Shiba Y, Fernandes S, Zhu W Z et al. Human ES-cell-derived cardiomyocytes electrically couple and suppress arrhythmias in injured hearts. Nature 2012; 489:322-5.
6. van Laake L W, Passier R, Monshouwer-Kloots J et al. Human embryonic stem cell-derived cardiomyocytes survive and mature in the mouse heart and transiently improve function after myocardial infarction. Stem Cell Res 2007; 1:9-24.
7. Shiba Y, Filice D, Fernandes S et al. Electrical Integration of Human Embryonic Stem Cell-Derived Cardiomyocytes in a Guinea Pig Chronic Infarct Model. J Cardiovasc Pharmacol Ther 2014; 19:368-381.
8. Chong J J, Yang X, Don C W et al. Human embryonic-stem-cell-derived cardiomyocytes regenerate non-human primate hearts. Nature 2014; 510:273-7.
9. Liu Y W, Chen B, Yang X et al. Human embryonic stem cell-derived cardiomyocytes restore function in infarcted hearts of non-human primates. Nat Biotechnol 2018; 36:597-605.
10. Shiba Y, Gomibuchi T, Seto T et al. Allogeneic transplantation of iPS cell-derived cardiomyocytes regenerates primate hearts. Nature 2016; 538:388-391.
11. Romagnuolo R, Masoudpour H, Porta-Sanchez A et al. Human Embryonic Stem Cell-Derived Cardiomyocytes Regenerate the Infarcted Pig Heart but Induce Ventricular Tachyarrhythmias. Stem Cell Reports 2019; 12:967-981.
12. Eschenhagen T, Bolli R, Braun T et al. Cardiomyocyte Regeneration: A Consensus Statement. Circulation 2017; 136:680-686.
13. Lelovas P P, Kostomitsopoulos N G, Xanthos T T. A comparative anatomic and physiologic overview of the porcine heart. J Am Assoc Lab Anim Sci 2014; 53:432-8.
14. van der Spoel TI, Jansen of Lorkeers S J, Agostoni P et al. Human relevance of pre-clinical studies in stem cell therapy: systematic review and meta-analysis of large animal models of ischaemic heart disease. Cardiovasc Res 2011; 91:649-58.
15. Chen V C, Ye J, Shukla P et al. Development of a scalable suspension culture for cardiac differentiation from human pluripotent stem cells. Stem Cell Res 2015; 15:365-75.
16. Staubli M, Bircher J, Galeazzi R L, Remund H, Studer H. Serum concentrations of amiodarone during long term therapy. Relation to dose, efficacy and toxicity. Eur J Clin Pharmacol 1983; 24:485-94.
17. Mostow N D, Rakita L, Vrobel T R, Noon D L, Blumer J. Amiodarone: correlation of serum concentration with suppression of complex ventricular ectopic activity. Am J Cardiol 1984; 54:569-74.
18. Schindelin J, Rueden C T, Hiner M C, Eliceiri K W. The ImageJ ecosystem: An open platform for biomedical image analysis. Mol Reprod Dev 2015; 82:518-29.
19. Deroulers C, Ameisen D, Badoual M, Gerin C, Granier A, Lartaud M. Analyzing huge pathology images with open source software. Diagn Pathol 2013; 8:92.
20. Garcia-Bustos V, Sebastian R, Izquierdo M, Molina P, Chorro F J, Ruiz-Sauri A. A quantitative structural and morphometric analysis of the Purkinje network and the Purkinje-myocardial junctions in pig hearts. J Anat 2017; 230:664-678.
21. Panescu D, Kroll M, Brave M. Limitations of animal electrical cardiac safety models. Annu Int Conf IEEE Eng Med Biol Soc 2014; 2014:6483-6.
22. Pallante B A, Giovannone S, Fang-Yu L et al. Contactin-2 expression in the cardiac Purkinje fiber network. Circ Arrhythm Electrophysiol 2010; 3:186-94.
23. Yu J K, Franceschi W, Huang Q, Pashakhanloo F, Boyle P M, Trayanova N A. A comprehensive, multiscale framework for evaluation of arrhythmias arising from cell therapy in the whole post-myocardial infarcted heart. Sci Rep 2019; 9:9238.
24. Chow E, Woodard J C, Farrar D J. Rapid ventricular pacing in pigs: an experimental model of congestive heart failure. Am J Physiol 1990;258:H1603-5.
25. Josephson M, Marchlinski F, Buxton A et al. Electrophysiologic basis for sustained ventricular tachycardia: role of reentry. Tachycardias: Mechanisms, Diagnosis, Treatment Philadelphia, Pa: Lea and Febiger 1984:305-323.
26. Karbassi E, Fenix A, Marchiano S et al. Cardiomyocyte maturation: advances in knowledge and implications for regenerative medicine. Nat Rev Cardiol 2020.
27. Waks J W, Zimetbaum P. Antiarrhythmic Drug Therapy for Rhythm Control in Atrial Fibrillation. J Cardiovasc Pharmacol Ther 2017; 22:3-19.
28. Kadota S, Pabon L, Reinecke H, Murry C E. In Vivo Maturation of Human Induced Pluripotent Stem Cell-Derived Cardiomyocytes in Neonatal and Adult Rat Hearts. Stem Cell Reports
29. Marchiano S, Bertero A, Murry C E. Learn from Your Elders: Developmental Biology Lessons to Guide Maturation of Stem Cell-Derived Cardiomyocytes. Pediatr Cardiol 2019; 40:1367-1387.
30. Guo Y, Pu W T. Cardiomyocyte Maturation: New Phase in Development. Circ Res 2020; 126:1086-1106.
31. Kannan S, Kwon C. Regulation of cardiomyocyte maturation during critical perinatal window. J Physiol 2020; 598:2941-2956.
32. Maroli G, Braun T. The long and winding road of cardiomyocyte maturation. Cardiovasc Res 2020.
33. Ichimura H, Kadota S, Kashihara T et al. Increased predominance of the matured ventricular subtype in embryonic stem cell-derived cardiomyocytes in vivo. Sci Rep 2020; 10:11883.
34. El-Nachef D, Oyama K, Wu Y Y, Freeman M, Zhang Y, MacLellan W R. Repressive histone methylation regulates cardiac myocyte cell cycle exit. J Mol Cell Cardiol 2018; 121:1-12.

1. A method of treating or ameliorating an engraftment arrhythmia in a subject recipient of a cardiac graft of cardiomyocytes, the method comprising administering to the subject an effective amount of amiodarone and an effective amount of ivabradine.

2. The method of claim 1, wherein the cardiac graft of cardiomyocytes comprises in vitro-differentiated cardiomyocytes.

3. The method of claim 2, wherein the in vitro-differentiated cardiomyocytes are differentiated from induced pluripotent stem (iPS) cells or from embryonic stem (ES) cells.

4. The method of claim 1, wherein the cardiac graft of cardiomyocytes is derived from stem cells autologous or allogeneic to the subject.

5. (canceled)

6. The method of claim 1, wherein the amiodarone and ivabradine are administered concurrently with the cardiac graft of cardiomyocytes.

7. The method of claim 1, wherein administration of amiodarone or ivabradine is initiated prior to administration of the cardiac graft of cardiomyocytes.

8-9. (canceled)

10. The method of claim 1, wherein the administration of ivabradine or amiodarone is initiated concurrently with or after administration of the cardiac graft of cardiomyocytes.

11. (canceled)

12. The method of claim 1, wherein the administration of amiodarone is a single bolus administration.

13. The method of claim 1, wherein the administration is:
(a) continuous or repeated administration, or
(b) oral administration and/or intravenous injection.

14. (canceled)

15. The method of claim 1, wherein the amiodarone is administered:
(A) orally at a dose of 100-800 mg, three times per days;
(b) by IV bolus at a dose of 100-300 mg; or
(c) to a serum concentration of 1.5 to 2.5 μg/ml.

16-17. (canceled)

18. The method of claim 1, wherein the ivabradine is orally administered at a dose of 5 to 15 mg, twice per day.

19. The method of claim 1, wherein the ivabradine is administered
(a) when there is tachycardia; or
(b) to maintain a resting heart rate of less than or equal to 150 beats per minute (bpm).

20. (canceled)

21. The method of claim 1, wherein the administration of amiodarone and ivabradine reduces post-graft accelerated heart rate experienced by the graft recipient by at least 10% relative to a subject receiving a graft of the same type of cells in the absence of amiodarone and ivabradine administration.

22. The method of claim 1, wherein the administration of amiodarone and ivabradine reduces the proportion of time in which the subject experiences engraftment arrhythmia by at least 10% relative to a subject receiving a cardiac graft of cardiomyocytes of the same type of cardiomyocytes in the absence of amiodarone and ivabradine administration.

23. The method of claim 1, wherein administration of amiodarone and ivabradine is:
(a) short-term; or
(b) terminated after engraftment arrhythmia burden reaches zero, without recrudescence of the arrhythmia.

24. (canceled)

25. A method of cardiomyocyte transplant, the method comprising:
a) administering in vitro-differentiated cardiomyocytes to cardiac tissue of a subject in need thereof;
b) administering to the subject an amount of amiodarone and an amount of ivabradine effective to reduce engraftment arrhythmia in the subject.

26-46. (canceled)

47. The method of claim 1, wherein about 10 million cardiomyocytes to about 10 billion cardiomyocytes are administered to the subject.

48. The method of claim 1, wherein the subject is a human.

49. The method of claim 1, wherein the subject has or is at risk for having a cardiovascular disease or a cardiac event selected from the group consisting of: atherosclerotic heart disease, myocardial infarction, cardiomyopathy, cardiac arrhythmia, valvular stenosis, congenital heart disease, chronic heart failure, regurgitation, ischemia, fibrillation, and polymorphic ventricular tachycardia.

50. (canceled)

51. A composition comprising in vitro-differentiated cardiomyocytes, amiodarone and ivabradine.
